SEQUENCE LISTING (54 sequences)

SEQ ID NO: 1 - DNA, 12 bp, Artificial Sequence
  Synthetic oligonucleotide primer
ggagatagaacc

SEQ ID NO: 2 - DNA, 11 bp, Artificial Sequence
  Synthetic oligonucleotide primer
gaaagctgggt

SEQ ID NO: 3 - DNA, 25 bp, Artificial Sequence
  Synthetic oligonucleotide primer
acaagtttgtacaaaaaagcaggct

SEQ ID NO: 4 - DNA, 46 bp, Artificial Sequence
  Synthetic oligonucleotide primer
ggggacaagtttgtacaaaaaagcaggcttcgaaggagatagaacc

SEQ ID NO: 5 - DNA, 25 bp, Artificial Sequence
  Synthetic oligonucleotide primer
accactttgtacaagaaagctgggt

SEQ ID NO: 6 - DNA, 30 bp, Artificial Sequence
  Synthetic oligonucleotide primer
ggggaccactttgtacaagaaagctgggtc

SEQ ID NO: 7 - DNA, 33 bp, Artificial Sequence
  Synthetic oligonucleotide primer
ggggaccactttgtacaagaaagctgggtctta

SEQ ID NO: 8 - DNA, 33 bp, Artificial Sequence
  Synthetic oligonucleotide primer
ggggaccactttgtacaagaaagctgggtcata

SEQ ID NO: 9 - DNA, 33 bp, Artificial Sequence
  Synthetic oligonucleotide primer
ggggaccactttgtacaagaaagctgggtctca

SEQ ID NO: 10 - DNA, 33 bp, Artificial Sequence
  Synthetic oligonucleotide primer
ggggaccactttgtacaagaaagctgggtcaca

SEQ ID NO: 11 - DNA, 33 bp, Artificial Sequence
  Synthetic oligonucleotide primer
ggggaccactttgtacaagaaagctgggtctcc

SEQ ID NO: 12 - DNA, 29 bp, Artificial Sequence
  Synthetic oligonucleotide primer
ggggaccactttgtacaagaaagctgggt

SEQ ID NO: 13 - protein, 4 aa, Unknown
  Factor Xa cleavage site
IEGR

SEQ ID NO: 14 - protein, 4 aa, Unknown
  Thrombin cleavage site
LVPR

SEQ ID NO: 15 - DNA, 25 bp, Unknown
  mutated att site attB1
agcctgcttttttgtacaaacttgt

SEQ ID NO: 16 - DNA, 233 bp, Unknown
  Mutated att site attP1
tacaggtcactaataccatctaagtagttgattcatagtgactggatatgttgtgttttacagtattatgtagtctgttttttatgcaaaatctaatttaatatattgatatttatatcattttacgtttctcgttcagcttttttgtacaaagttggcattataaaaaagcattgctcatcaatttgttgcaacgaacaggtcactatcagtcaaaataaaatcattatttg

SEQ ID NO: 17 - DNA, 100 bp, Unknown
  Mutated att site attL1
caaataatgattttattttgactgatagtgacctgttcgttgcaacaaattgataagcaatgcttttttataatgccaactttgtacaaaaaagcaggct

SEQ ID NO: 18 - DNA, 125 bp, Unknown
  Mutated att site attR1
acaagtttgtacaaaaaagctgaacgagaaacgtaaaatgatataaatatcaatatattaaattagattttgcataaaaaacagactacataatactgtaaaacacaacatatccagtcactatg

SEQ ID NO: 19 - DNA, 27 bp, Unknown
  wild type att site attB0
agcctgcttttttatactaacttgagc

SEQ ID NO: 20 - DNA, 27 bp, Unknown
  wild type att site attP0
gttcagcttttttatactaagttggca

SEQ ID NO: 21 - DNA, 27 bp, Unknown
  wild type att site attL0
agcctgcttttttatactaagttggca

SEQ ID NO: 22 - DNA, 27 bp, Unknown
  wild type att site attR0
gttcagcttttttatactaacttgagc

SEQ ID NO: 23 - DNA, 25 bp, Unknown
  mutated att site attB1
agcctgcttttttgtacaaacttgt

SEQ ID NO: 24 - DNA, 27 bp, Unknown
  mutated att site attP1
gttcagcttttttgtacaaagttggca

SEQ ID NO: 25 - DNA, 27 bp, Unknown
  mutated att site attL1
agcctgcttttttgtacaaagttggca

SEQ ID NO: 26 - DNA, 25 bp, Unknown
  mutated att site attR1
gttcagcttttttgtacaaacttgt

SEQ ID NO: 27 - DNA, 25 bp, Unknown
  mutated att site attB2
acccagctttcttgtacaaagtggt

SEQ ID NO: 28 - DNA, 27 bp, Unknown
  mutated att site attP2
gttcagctttcttgtacaaagttggca

SEQ ID NO: 29 - DNA, 27 bp, Unknown
  mutated att site attL2
acccagctttcttgtacaaagttggca

SEQ ID NO: 30 - DNA, 25 bp, Unknown
  mutated att site attR2
gttcagctttcttgtacaaagtggt

SEQ ID NO: 31 - DNA, 22 bp, Unknown
  mutated att site attB5
caactttattatacaaagttgt

SEQ ID NO: 32 - DNA, 27 bp, Unknown
  mutated att site attP5
gttcaactttattatacaaagttggca

SEQ ID NO: 33 - DNA, 24 bp, Unknown
  mutated att site attL5
caactttattatacaaagttggca

SEQ ID NO: 34 - DNA, 25 bp, Unknown
  mutated att site attR5
gttcaactttattatacaaagttgt

SEQ ID NO: 35 - DNA, 22 bp, Unknown
  mutated att site attB11
caacttttctatacaaagttgt

SEQ ID NO: 36 - DNA, 27 bp, Unknown
  mutated att site attP11
gttcaacttttctatacaaagttggca

SEQ ID NO: 37 - DNA, 24 bp, Unknown
  mutated att site attL11
caacttttctatacaaagttggca

SEQ ID NO: 38 - DNA, 25 bp, Unknown
  mutated att site attR11
gttcaacttttctatacaaagttgt

SEQ ID NO: 39 - DNA, 22 bp, Unknown
  mutated att site attB17
caacttttgtatacaaagttgt

SEQ ID NO: 40 - DNA, 27 bp, Unknown
  mutated att site attP17
gttcaacttttgtatacaaagttggca

SEQ ID NO: 41 - DNA, 24 bp, Unknown
  mutated att site attL17
caacttttgtatacaaagttggca

SEQ ID NO: 42 - DNA, 25 bp, Unknown
  mutated att site attR17
gttcaacttttgtatacaaagttgt

SEQ ID NO: 43 - DNA, 22 bp, Unknown
  mutated att site attB19
caactttttcgtacaaagttgt

SEQ ID NO: 44 - DNA, 27 bp, Unknown
  mutated att site attP19
gttcaactttttcgtacaaagttggca

SEQ ID NO: 45 - DNA, 24 bp, Unknown
  mutated att site attL19
caactttttcgtacaaagttggca

SEQ ID NO: 46 - DNA, 25 bp, Unknown
  mutated att site attR19
gttcaactttttcgtacaaagttgt

SEQ ID NO: 47 - DNA, 22 bp, Unknown
  mutated att site attB20
caactttttggtacaaagttgt

SEQ ID NO: 48 - DNA, 27 bp, Unknown
  mutated att site attP20
gttcaactttttggtacaaagttggca

SEQ ID NO: 49 - DNA, 24 bp, Unknown
  mutated att site attL20
caactttttggtacaaagttggca

SEQ ID NO: 50 - DNA, 25 bp, Unknown
  mutated att site attR20
gttcaactttttggtacaaagttgt

SEQ ID NO: 51 - DNA, 22 bp, Unknown
  mutated att site attB21
caactttttaatacaaagttgt

SEQ ID NO: 52 - DNA, 27 bp, Unknown
  mutated att site attP21
gttcaactttttaatacaaagttggca

SEQ ID NO: 53 - DNA, 24 bp, Unknown
  mutated att site attL21
caactttttaatacaaagttggca

SEQ ID NO: 54 - DNA, 25 bp, Unknown
  mutated att site attR21
gttcaactttttaatacaaagttgt

(19) United States
(12) Patent Application Publication
(10) Pub. No.: US 2004/0040053 A1
(43) Pub. Date: Feb. 26, 2004

(54) Method for the preparation of nucleic acid

(76) Inventors: Nobuo Nomura, Tokyo (JP); Naoki Goshima, Tokyo (JP); Yasutomo Kisu, Chiba (JP); Saki Sono, Chiba (JP)

Correspondence Address: STERNE, KESSLER, GOLDSTEIN & FOX PLLC, 1100 NEW YORK AVENUE, N.W., WASHINGTON, DC, 20005, US

(21) Appl. No.: 10/301,849

(22) Filed: Nov. 22, 2002

(30) Foreign Application Priority Data
Nov. 22, 2001 (JP) 2001-357821

(51) Int. Cl. C12P 19/34; A01H 1/00; C12N 15/82

(52) U.S. Cl. 800/278; 435/91.2

(57) ABSTRACT

The present invention provides materials and methods for the rapid cloning and mutagenesis of nucleic acid molecules. The present invention permits simultaneous introduction of a one or more point mutations and adapter sequences into a nucleic acid of interest.

### BACKGROUND OF THE INVENTION

1. Field of the Invention

The present invention relates to the field of genetic engineering and, more particularly, to the amplification, mutagenesis and cloning of nucleic acids.

2. Related Art

Conventional Nucleic Acid Cloning

The cloning of nucleic acid segments currently occurs as a daily routine in many research labs and as a prerequisite step in many genetic analyses. The purpose of these clonings is various, however, two general purposes can be considered: (1) the initial cloning of nucleic acid from large DNA or RNA segments (chromosomes, YACs, PCR fragments, mRNA, etc.), done in a relative handful of known vectors such as pUC, pGem, pBlueScript, and (2) the subcloning of these nucleic acid segments into specialized vectors for functional analysis. A great deal of time and effort is expended both in the transfer of nucleic acid segments from the initial cloning vectors to the more specialized vectors. This transfer is called subcloning.

The basic methods for cloning have been known for many years and have changed little during that time. A typical cloning protocol is as follows:

(1) digest the nucleic acid of interest with one or two restriction enzymes;

(2) gel purify the nucleic acid segment of interest when known;

(3) prepare the vector by cutting with appropriate restriction enzymes, treating with alkaline phosphatase, gel purify etc., as appropriate;

(4) ligate the nucleic acid segment to the vector, with appropriate controls to eliminate background of uncut and self-ligated vector;

(5) introduce the resulting vector into an E. coli host cell;

(6) pick selected colonies and grow small cultures overnight;

(7) make nucleic acid minipreps; and

(8) analyze the isolated plasmid on agarose gels (often after diagnostic restriction enzyme digestions) or by PCR.

The specialized vectors used for subcloning nucleic acid segments are functionally diverse. These include but are not limited to: vectors for expressing nucleic acid molecules in various organisms; for regulating nucleic acid molecule expression; for providing tags to aid in protein purification or to allow tracking of proteins in cells; for modifying the cloned nucleic acid segment (e.g., generating deletions); for the synthesis of probes (e.g., riboprobes); for the preparation of templates for nucleic acid sequencing; for the identification of protein coding regions; for the fusion of various protein-coding regions; to provide large amounts of the nucleic acid of interest, etc. It is common that a particular investigation will involve subcloning the nucleic acid segment of interest into several different specialized vectors.

As known in the art, simple subclonings can be done in one day (e.g., the nucleic acid segment is not large and the restriction sites are compatible with those of the subcloning vector). However, many other subclonings can take several weeks, especially those involving unknown sequences, long fragments, toxic genes, unsuitable placement of restriction sites, high backgrounds, impure enzymes, etc. One of the most tedious and time consuming type of subcloning involves the sequential addition of several nucleic acid segments to a vector in order to construct a desired clone. One example of this type of cloning is in the construction of gene targeting vectors. Gene targeting vectors typically include two nucleic acid segments, each identical to a portion of the target gene, flanking a selectable marker. In order to construct such a vector, it may be necessary to clone each segment sequentially, i.e., first one gene fragment is inserted into the vector, then the selectable marker and then the second fragment of the target gene. This may require a number of digestion, purification, ligation and isolation steps for each fragment cloned. Subcloning nucleic acid fragments is thus often viewed as a chore to be done as few times as possible. Considerable labor is expended, and if two or more days later the desired subclone can not be found among the candidate plasmids, the entire process must then be repeated with alternative conditions attempted.

Recombinational Cloning

Cloning systems that utilize recombination at defined recombination sites have been previously described in U.S. Pat. Nos. 5,888,732, 6,143,557, 6,171,861, 6,270,969, and 6,277,608 which are specifically incorporated herein by reference. A commercial embodiment of the methods described in these patents (the GATEWAY™ Cloning System, Invitrogen Corporation, Carlsbad, Calif.), utilizes vectors that contain at least one and preferably at least two different site-specific recombination sites based on the bacteriophage lambda system (e.g., att1 and att2) that are mutated from the wild type (att0) sites. Each mutated site has a unique specificity for its cognate partner att site of the same type (for example attB1 with attP1, or attL1 with attR1) and will not cross-react with recombination sites of the other mutant type or with the wild-type att0 site. Nucleic acid fragments flanked by recombination sites are cloned and subcloned using the GATEWAY™ system by replacing a selectable marker (for example, ccdB) flanked by att sites on the recipient plasmid molecule, sometimes termed the Destination Vector. Desired clones are then selected by transformation of a ccdB sensitive host strain and positive selection for a marker on the recipient molecule. Similar strategies for negative selection (e.g., use of toxic genes) can be used in other organisms such as thymidine kinase (TK) in mammals and insects.

A GATEWAY™ cloning technology is a rapid and highly efficient general-use cloning system for analyzing functions of cDNAs and PCR DNAS, expressing proteins, and cloning or sub-cloning (U.S. Pat. No. 5,888,732). This GATEWAY™ system is the technology of in vitro carrying out crossover of an insert DNA between different vectors for a short period of time without using a restriction enzyme or a ligase by utilizing a site-specific DNA recombination reaction, thereby constructing an entry clone as a base and forming a variety of expressed clones at a high speed and with ease.

This GATEWAY™ system can form an entry clone by first inserting a desired genomic DNA into an entry vector. The entry clone can also be formed by PCR, a restriction enzyme digestion or a ligase connection. Further, it is known that it can be formed by means of a site-specific recombination reaction from a cDNA library constructed with a GATEWAY™ vector. A desired DNA connected to a recombination site called an att site is mixed with an enzymatic mix of an appropriate destination vector with a clonase, first forming a co-integrate molecule comprising both starting molecules and then resolving the co-integrate molecule into two new molecules. From one of these two molecules, a new vector molecule (an expression clone) containing the desired DNA can be isolated. The other molecule contains a gene for a negative selection (ccdB) so that a host organism is killed to allow an efficient recovery (90% or higher) of the desired clone by means of the positive and negative selections.

Topoisomerase-Mediated Cloning

U.S. Pat. No. 5,766,891 issued to Shuman describes methods of using a topoisomerase (e.g., vaccinia virus topoisomerase), to clone nucleic acids. Such methods typically entail adding a topoisomerase recognition site to a nucleic acid molecule of interest, reacting the recognition site with a topoisomerase enzyme to produce a covalent intermediate comprising the topoisomerase and the nucleic acid of interest and reacting the covalent intermediate with a suitable vector. In other configurations, the vector can be equipped with a topoisomerase recognition sequence and reacted with an nucleic acid of interest. In yet other configurations, both the vector and the nucleic acid of interest may be equipped with topoisomerase recognition sites. Methods of cloning using topoisomerase are commercially available from Invitrogen Corporation, Carlsbad, Calif. Methods employing both recombinational cloning and topoisomerase-mediated cloning in conjunction have also been described (see WO 02/46372). Other methods are described in U.S. provisional patent application serial No. 60/385,613, filed Jun. 5, 2002, which is specifically incorporated herein by reference.

Mutagenesis

After cloning a nucleic acid of interest, it is often desirable to change the nucleotide sequence of the cloned nucleic acid. The process of changing the nucleotide sequence is referred to as mutagenesis. While it is sometimes desirable to randomly change the nucleotide sequence, for most applications, it is preferable to introduce changes in the sequence at known positions. The process of changing the nucleotide sequence at specific sites is frequently referred to as site-directed mutagenesis.

Numerous techniques have been developed to conduct site-directed mutagenesis. One of the most frequently used employs the polymerase chain reaction (PCR). Typically, the sequence of one or more of the primers to be used in the amplification is selected so as to differ from the template sequence at one or more positions. Multiple rounds of primer extension and denaturation are performed resulting in a PCR product that incorporates the sequence of the primer. Numerous variations of mutagenesis methods employing PCR are known (see, for example, Tao, B. and Lee, P. “Mutagenesis by PCR” in PCR Technology: Current Innovations, Griffin, H. and Griffin, A., eds. CRC Press, Inc., Ann Arbor, Mich., Chapter 10, pp. 69-83, 1994).

Notwithstanding the methods described above, there remains in the art a need for more efficient methods for cloning and mutagenizing nucleic acids. This need and others are met by the present invention.

### SUMMARY OF THE INVENTION

The present invention provides materials and methods for the preparation of nucleic acid molecules. In some embodiments, the present invention provides a method of preparing a plurality of nucleic acid molecules, by contacting a template nucleic acid molecule with at least a first, second and third primer and a polypeptide having DNA polymerase activity to form a mixture and incubating the mixture under conditions sufficient to extend the primers, wherein both the second and the third primers comprise a sequence that anneals to a sequence on the template and, wherein the sequence of at least one of the second and third primer comprises at least one base (e.g., two, three, four, five, etc.) that does not base pair with the sequence of the template. In some embodiments, the plurality of nucleic acids may comprise nucleic acid molecules that contain sequences of interest that differ from each other by the presence or absence of a specific nucleotide at a specific location. For example, the plurality of nucleic acids may comprise a first nucleic acid having a particular codon at specified location and another nucleic acid in the plurality may differ from the first in that one nucleotide of the codon (e.g., the last nucleotide) may be different. In a particular embodiment, a first nucleic acid may have a stop codon at a particular location and a second nucleic acid may have a codon at the same position that codes for an amino acid by virtue of having one nucleotide changed relative to the stop codon. Any of the nucleotides of a codon may be changed. Thus, the invention provides, in part, methods for preparing populations of nucleic acid molecules that differ in nucleotide sequence. The invention also provides populations nucleic acid molecules prepared by methods described herein, as well as compositions comprising these populations of nucleic acid molecules.

In some aspects, the present invention provides a method of amplifying a double-stranded DNA molecule comprising:

(a) providing at least a first, second and third primer, wherein the first primer is at least partially complementary to a sequence of the second strand of the DNA molecule and the second and third primers are at least partially complementary to a sequence of the first strand of the DNA molecule, in many instances at least one of the second and third primers may contain at least one nucleotide that does not base pair to the sequence on the first strand of the DNA molecule to which it hybridizes; and

(b) hybridizing the first primer to the second strand and the second and third primers to the first strand in the presence of a polypeptide having DNA polymerase activity, under conditions such that a third DNA strand complementary to the second strand and a fourth and a fifth DNA strand complementary to the first strand are synthesized. Typically, the second and third primers contain a sequence that hybridizes to the same sequence on the template molecule. The portion of the second and third primers that hybridizes to the template may differ in nucleotide sequence by one or more nucleotides. The nucleotides that differ from exact complementarity with the template may be located anywhere in the portion of the sequence of the primer that hybridizes to the template. In some embodiments, the nucleotides that differ from exact complementarity may be located near the 3′-terminus of the primer. In a particular embodiment, a primer may comprise a single nucleotide that differs from exact complementarity with the template and this nucleotide may be located two nucleotides from the 3′-most nucleotide of the primer.

Methods of this type may further comprise:

(c) denaturing the product of (b); and

(d) repeating (a) to (c) one or more times to produce third strands comprising a sequence complementary to either the second or the third primer and to produce fourth and fifth strands comprising a sequence complementary to the first primer. The number of times that (a) to (c) may be repeated may range from 1 to about 50, from 1 to about 25, from 1 to about 15, from 1 to about 10, from 1 to about 8, from 1 to about 5, from about 3 to about 50, from about 3 to about 25, from about 3 to about 15, from about 3 to about 10, from about 3 to about 8, or from about 3 to about 5.

In some embodiments of the invention, one or more of primer one, two or three may comprise an adapter sequence. For example, the first primer may comprise a first adapter sequence, the second primer may comprise a second adapter sequence and/or the third primer may comprise a third adapter sequence. The adapter sequences may be the same or different. In some embodiments, the adapter sequence of the second and the third primer are the same whereas in other embodiments the adapter sequence of the second and the third primer are different.

In some embodiments, methods of the present invention may include:

(e) contacting the product of (d) with at least a fourth, fifth and sixth primer, wherein the fourth primer is at least partially complementary to the fourth and fifth strands and the fifth and sixth primers are at least partially complementary to the third strands; and

(f) hybridizing the fourth primer to the fourth and fifth strands and the fifth and sixth primers to the third strands in the presence of a polypeptide having DNA polymerase activity, under conditions such that a sixth DNA strand complementary to the fourth strand, a seventh DNA strand complementary to the fifth strand, an eighth DNA strand complementary to the thirds strands, and a ninth DNA strand complementary to the third strands are synthesized. Methods of this type may further include:

(g) denaturing the product of (f); and

(h) repeating (e) to (g) one or more times to produce sixth strands comprising a sequence complementary to the fifth primer, seventh strands comprising a sequence complementary to the sixth primer, eighth strands comprising a sequence complementary to the fourth primer and ninth strands comprising a sequence complementary to the fourth primer. Primers that may be used in methods of this type may comprise adapter sequences. For example, one or more of the fourth, fifth or sixth primers may comprise an adapter sequence. For example, the fourth primer may. comprise a fourth adapter sequence, the fifth primer may comprise a fifth adapter sequence, and/or the sixth primer may comprise a sixth primer sequence. In some embodiments, the adapter sequences of the fifth and sixth primers are the same. In other embodiments, the adapter sequences of the fifth and sixth primers are different. Adapter sequences that may be included in primers for use in the invention included any sequence or sequences known to those of skille in the art. Adapter sequences include, but are not limited to, recognition sequences. In some embodiments, one or more adapter sequence may comprise a sequence independently selected from the group consisting of restriction enzyme recognition sites, topoisomerase recognition sites, recombination sites, transposition sites, coding sequences (e.g., sequences encoding peptide tags such as 6-histidines, the V5 epitope, etc.), transcriptional and/or translational regulatory sequences (e.g., promoter sequences, enhancer sequences, repressor sequences, Shine-Dalgarno sequences, Kozak sequences, etc.) as well as other sequences that can be used in processes such as molecular cloning and recombination. Recombination site sequences that may be included in adapters may be any recombination site sequence known to those skilled in the art. In some embodiments, the recombination site sequences may be selected from a group consisting of lox sites and att sites. Examples of suitable att sites include, but are not limited to, attB1, attP1, attL1, attR1, attB2, attP2, attL2, attR2, attB3, attP3, attL3, attR3, attB5, attP5, attL5, and attR5.

Nucleic acid molecules prepared as described above may be inserted and/or cloned into one or more vectors. Accordingly, methods of the invention may include:

(i) contacting the product of (h) with one or more polypeptides and one or more vectors under conditions sufficient to insert all or a portion of the product of (h) into the vector. Such a method may also include:

(1) transforming a competent cell with the product of (i); and

(k) selecting for cells comprising the product of (i). Any vector known to those skilled in the art may be used to practice this aspect of the invention. For example, one or more vectors used in this aspect of the invention may comprise at least one recombination site. Typically, when one or more vectors used in this aspect of the invention comprise a recombination site, the product of (h) is contacted with the recombination-site-containing vector and at least one recombination protein as well as any other polypeptides that may be required. In some embodiments, methods according to the present invention may comprise contacting the product of (h) with one or more polypeptides (e.g., recombination proteins, topoisomerases, restriction enzymes, ligases, etc.) and at least two different vectors under conditions sufficient to insert all or a portion of the product of (h) into the vectors. Methods of the invention may also include selecting for one or more of the vectors containing a desired insert. In some embodiments, vectors containing the product of (h) may be selected using different selection schemes, i.e., the selection scheme used to select a first vector is different from the selection scheme used to select a second vector.

In a specific embodiment, the PCR method for the preparation of an entry clone may involve a PCR amplification of a desired genomic DNA with two kinds of primers with adapters each having a recombination sequence attB1 of 25 bp described in SEQ ID NO. 3 at 5′-terminus and a recombination sequence attB2 of 25 bp described in SEQ ID NO. 5 at 3′-terminus, thereby yielding an adapter-containing PCR product. If the desired genomic DNA is to be transferred to an attP plasmid, it can be transferred into the plasmid by means of an in vitro BP reaction while maintaining the directionality of the desired DNA (GATEWAY™ Cloning Technology (2000), Naoki Goto, Yasutomo Kisu and Fumio Imamoto, Experimental Medical Science, 18(19) 2716-2717).

Making the formation of the entry clone by the PCR method more efficient is the significant object for making the GATEWAY™ system more useful. In particular, the development for a selective formation of an expressed clone of a fused protein is greatly demanded as a technology of significance for enlarging the general usability and the scope of application of the GATEWAY™ system.

The present invention provides a technology for the selective formation of a GATEWAY™ entry clone of a native-type cDNA and a C-terminal fused-type cDNA.

As a result of extensive review on the ways to achieve the above-mentioned object, the present invention has been completed.

The present invention provides the methods as will be described hereinafter.

In some embodiments, the present method provides, a method for the preparation of a GATEWAY™ entry clone by a two-step adapter PCR method. With reference to FIG. 3, a first PCR is conducted in which a template (e.g., a cDNA) is amplified by using a 5′-terminal-primer A containing an adapter a and a mixture of 3′-terminal primers B and B′ having a different sequence of bases at a site hybridizable with a stop codon of the template cDNA and containing an adapter b. A second PCR may be performed in which an amplified product obtained by the first PCR step is amplified as a template by using a 5′-terminal common primer C having an adapter c, which adapter sequence may comprise a sequence attB1 (e.g., at the 5′-terminus of the primer), which sequence may act as a site connectable to a plasmid, and a native-type common cDNA primer D or a C-terminal fused-type common primer D′ as a 3′-terminal primer, each having an adapter d, which may comprise a sequence attB2 (e.g., at the 5′-terminus of the primer), which sequence may act as a site connectable to the plasmid, thereby forming a native-type cDNA or a C-terminal fused-type cDNA each of which may have different sequences of bases at both termini. The resultant cDNA may be inserted into a plasmid (e.g., into an attP plasmid) and the resultant plasmid may be introduced into a host cell.

The present invention also contemplates a PCR primer C as described above, comprising a sequence of bases as described in SEQ ID NO. 4.

In some embodiments, methods of the invention may comprise the use of: a primer B comprising a sequence of bases 3′-ATT-5′ hybridizable with a stop codon on a template nucleic acid molecule; a primer B′ comprising a sequence of bases 3′-ATA-5′ hybridizable with a stop codon present on a nucleic acid template molecule; a primer D comprising a sequence of bases as described in SEQ ID NO. 7; and/or a primer D′ comprising a sequence of bases as described in SEQ ID NO. 8. The present invention also contemplates a PCR primer D comprising a sequence of bases as described in SEQ ID NO. 7 and contemplates a PCR primer D′ comprising a sequence of bases as described in SEQ ID NO. 8.

In some embodiments, methods of the invention may comprise the use of: a primer B comprising a sequence of bases 3′-ACT-5′ hybridizable with a stop codon on a template nucleic acid molecule; a primer B′ comprising a sequence of bases 3′-ACA-5′ hybridizable with a stop codon on a template nucleic acid molecule; a primer D comprising a sequence of bases as described in SEQ ID NO. 9; and/or a primer D′ comprising a sequence of bases as described in SEQ ID NO. 10. The present invention also contemplates a PCR primer D comprising a sequence of bases as described in SEQ ID NO. 9 and contemplates a PCR primer D′ comprising a sequence of bases as described in SEQ ID NO. 10.

In some embodiments, methods of the invention may comprise the use of: a primer B comprising a sequence of bases 3′-ACT-5′ hybridizable with a stop codon on a template nucleic acid molecule; the primer B′ comprises a sequence of bases 3′-CCT-5 hybridizable with a stop codon on a template nucleic acid molecule; a primer D comprising a sequence of bases as described in SEQ ID NO. 9; and/or a primer D′ comprising a sequence of bases as described in SEQ ID NO. 11. The present invention also contemplates a PCR primer D′ comprising a sequence of bases as described in SEQ ID NO. 11.

The present invention also contemplates GATEWAY™ entry clones of the native type cDNA and the C-terminal fused-type cDNA formed by methods described herein.

### BRIEF DESCRIPTION OF THE DRAWINGS/FIGURES

FIG. 1 is a schematic representation of a basic recombinational cloning reaction.

FIG. 2 is a schematic representation of methods of the invention. Primers are labeled P1 through P6, strands of nucleic acid are labeled S1 through S9. X indicates a particular site (e.g., a nucleotide) on the template to be mutagenized.

FIG. 3 is a schematic diagram showing the amplification of cDNA by the two-step adapter PCR in accordance with the present invention. ATG: sequence of initiation codon for reading, Ter: sequence of stop codon (general term for TGA, TAA and TAG), ORF: translation region of cDNA, W in the primer sequence is used to indicate the nucleotide in question may be A or T. The adapter sequence of the primer used to anneal to the ATG portion of the template may include transcription and/or translation control signals (e.g., a Shine-Delgarno sequence, a Kozak box sequence, etc.). Primers are indicated as capital letters and the adapter sequence of the primer is indicated as a lower case letter. For example, primer A has and adapter sequence a.

FIG. 4 is a schematic representation of one embodiment of the invention. A template molecule comprising a sequence of interest is amplified in two separate PCR reactions. The sequence of interest is exemplified as an open reading frame from ATG - - - Ter where Ter stand for a stop codon. In tube 1, primer D results in amplification of stop-codon-containing PCR product. In tube 2, primer D′ results in the amplification of PCR product in which the stop codon has been eliminated.

### DETAILED DESCRIPTION OF THE INVENTION

Definitions

In the description that follows, a number of terms used in recombinant nucleic acid technology are utilized extensively. In order to provide a clear and more consistent understanding of the specification and claims, including the scope to be given such terms, the following definitions are provided.

Host: As used herein, a host is any prokaryotic or eukaryotic organism that is a recipient of a replicable expression vector, cloning vector or any nucleic acid molecule. The nucleic acid molecule may contain, but is not limited to, a structural gene, a transcriptional regulatory sequence (such as a promoter, enhancer, repressor, and the like) and/or an origin of replication. As used herein, the terms “host,” “host cell,” “recombinant host” and “recombinant host cell” may be used interchangeably. For examples of such hosts, see Maniatis et al., Molecular Cloning: A Laboratory Manual, Cold Spring Harbor Laboratory, Cold Spring Harbor, N.Y. (1982).

Transcriptional Regulatory Sequence: As used herein, transcriptional regulatory sequence is a functional stretch of nucleotides contained on a nucleic acid molecule, in any configuration or geometry, that acts to regulate the transcription of one or more DNA sequences into RNA (e.g., mRNA). Examples of transcriptional regulatory sequences include, but are not limited to, promoters, operators, enhancers, repressors, and the like. Transcriptional regulatory sequences may also regulate the transcription of nucleic acid molecules which encode functional RNAs (e.g., ribozymes, tRNAs, rRNAs, mRNAs, etc.).

Promoter: As used herein, a promoter is an example of a transcriptional regulatory sequence, and is specifically a nucleic acid sequence generally described as the 5′-region of a gene located proximal to the start codon. The transcription of an adjacent nucleic acid segment is initiated at the promoter region. A repressible promoter's rate of transcription decreases in response to a repressing agent. An inducible promoter's rate of transcription increases in response to an inducing agent. A constitutive promoter's rate of transcription is not specifically regulated, though it can vary under the influence of general metabolic conditions.

Insert: As used herein, an insert is a desired nucleic acid segment that is a part of a larger nucleic acid molecule.

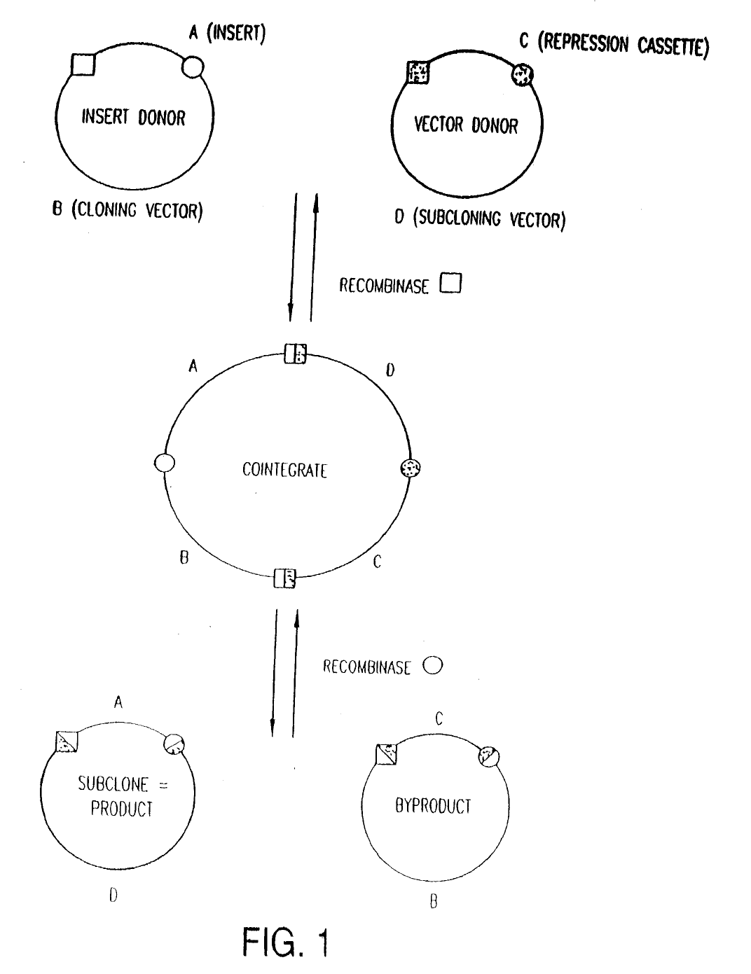

Insert Donor: As used herein, an insert donor is one of the two parental nucleic acid molecules (e.g. RNA or DNA) of the present invention which carries the Insert. The Insert Donor molecule comprises the Insert flanked on one or both sides with recombination sites. The Insert Donor can be linear or circular. In one embodiment of the invention, the Insert Donor is a circular nucleic acid molecule, optionally supercoiled, and further comprises a cloning vector sequence outside of the recombination signals (see FIG. 1). When a population of Inserts or population of nucleic acid segments are used to make the Insert Donor, a population of Insert Donors result and may be used in accordance with the invention.

Product: As used herein, a product is one the desired daughter molecules comprising the A and D sequences, which is produced after the second recombination event during the recombinational cloning process (see FIG. 1). The Product contains the nucleic acid which was to be cloned or subcloned (e.g., a nucleic acid sequence of interest). In accordance with the invention, when a population of Insert Donors are used, the resulting population of Product molecules will contain all or a portion of the population of Inserts of the Insert Donors and preferably will contain a representative population of the original molecules of the Insert Donors.

Recognition sequence: As used herein, a recognition sequence (alternatively and equivalently referred to herein as a “recognition site”) is a particular sequence to which a protein, chemical compound, DNA, or RNA molecule (e.g., restriction endonuclease, a topoisomerase, a modification methylase, or a recombinase) recognizes and binds. In the present invention, a recognition sequence may refer to a recombination site (which may alternatively be referred to as a recombinase recognition site) or a topoisomerase recognition site. For example, a recognition sequence for Cre recombinase is loxP. Recognition sequences for the lambda phage recombination enzyme Integrase include attB, attP, attL, and attR sequences. Examples of topoisomerase recognition sequences include, but are not limited to, the sequence 5′-GCAACTT-3′ that is recognized by E. coli topoisomerase III (a type I topoisomerase); the sequence 5′-(C/T)CCTT-3′, which is a topoisomerase recognition site that is bound specifically by most poxvirus topoisomerases, including vaccinia virus DNA topoisomerase I; and others that are known in the art as discussed elsewhere herein.

Recombination proteins: As used herein, recombination proteins include excisive or integrative proteins, enzymes, co-factors or associated proteins that are involved in recombination reactions involving one or more recombination sites, which may be wild-type proteins (See Landy, Current Opinion in Biotechnology 3:699-707 (1993)), or mutants, derivatives (e.g., fusion proteins containing the recombination protein sequences or fragments thereof), fragments, and variants thereof.

Recombination site: A used herein, a recombination site is a recognition sequence on a nucleic acid molecule participating in an integration/recombination reaction by recombination proteins. Recombination sites are discrete sections or segments of nucleic acid on the participating nucleic acid molecules that are recognized and bound by a site-specific recombination protein during the initial stages of integration or recombination. For example, the recombination site for Cre recombinase is loxP, which is a 34 base pair sequence comprised of two 13 base pair inverted repeats (serving as the recombinase binding sites) flanking an 8 base pair core sequence. See FIG. 1 of Sauer, B., Curr. Opin. Biotech. 5:521-527 (1994). Other examples of recognition sequences include the attB, attP, attL, and attR sequences described herein, and mutants, fragments, variants and derivatives thereof, which are recognized by the recombination protein Int and by the auxiliary proteins integration host factor (IIF), FIS and excisionase (Xis). See Landy, Curr. Opin. Biotech. 3:699-707 (1993). attB is an approximately 25 base pair sequence containing two 9 base pair core-type Int binding sites and a 7 base pair overlap region. attP is an approximately 240 base pair sequence containing core-type Int binding sites and arm-type Int binding sites as well as sites for auxiliary proteins integration host factor (IHF), FIS and excisionase (Xis). Such sites may also be engineered according to the present invention to enhance production of products in the methods of the invention. When such engineered sites lack the P1 or H1 domains to make the recombination reactions irreversible (e.g., attR or attP), such sites may be designated attR′ or attP to show that the domains of these sites have been modified in some way.

Recombinational Cloning: As used herein, recombinational cloning is a method, such as that described in U.S. Pat. Nos. 5,888,732, 6,143,557, 6,171,861, 6,270,969, and 6,277,608 (the contents of which are fully incorporated herein by reference), and as also described herein, whereby segments of nucleic acid molecules or populations of such molecules are exchanged, inserted, replaced, substituted or modified, in vitro or in vivo. Preferably, such cloning method is an in vitro method.

Repression cassette: As used herein, repression cassette is a nucleic acid segment that contains a repressor or a Selectable marker present in the subcloning vector.

Selectable marker: As used herein, selectable marker is a nucleic acid segment that allows one to select for or against a molecule (e.g., a replicon) or a cell that contains it, often under particular conditions. These markers can encode an activity, such as, but not limited to, production of RNA, peptide, or protein, or can provide a binding site for RNA, peptides, proteins, inorganic and organic compounds or compositions and the like. Examples of selectable markers include but are not limited to: (1) nucleic acid segments that encode products which provide resistance against otherwise toxic compounds (e.g., antibiotics); (2) nucleic acid segments that encode products which are otherwise lacking in the recipient cell (e.g., tRNA genes, auxotrophic markers); (3) nucleic acid segments that encode products which suppress the activity of a gene product; (4) nucleic acid segments that encode products which can be readily identified (e.g., phenotypic markers such as (β-galactosidase, green fluorescent protein (GFP), and cell surface proteins); (5) nucleic acid segments that bind products which are otherwise detrimental to cell survival and/or function; (6) nucleic acid segments that otherwise inhibit the activity of any of the nucleic acid segments described in Nos. 1-5 above (e.g., antisense oligonucleotides); (7) nucleic acid segments that bind products that modify a substrate (e.g. restriction endonucleases); (8) nucleic acid segments that can be used to isolate or identify a desired molecule (e.g. specific protein binding sites); (9) nucleic acid segments that encode a specific nucleotide sequence which can be otherwise non-functional (e.g., for PCR amplification of subpopulations of molecules); (10) nucleic acid segments, which when absent, directly or indirectly confer resistance or sensitivity to particular compounds; and/or (11) nucleic acid segments that encode products which are toxic in recipient cells.

Selection scheme: As used herein, selection scheme is any method which allows selection, enrichment, or identification of a desired Product or Product(s) from a mixture containing an Entry Clone or Vector, a Destination Vector, a Donor Vector, an Expression Clone or Vector, any intermediates (e.g. a Cointegrate or a replicon), and/or Byproducts. The selection schemes of one preferred embodiment have at least two components that are either linked or unlinked during recombinational cloning. One component is a Selectable marker. The other component controls the expression in vitro or in vivo of the Selectable marker, or survival of the cell (or the nucleic acid molecule, e.g., a replicon) harboring the plasmid carrying the Selectable marker. Generally, this controlling element will be a repressor or inducer of the Selectable marker, but other means for controlling expression or activity of the Selectable marker can be used. Whether a repressor or activator is used will depend on whether the marker is for a positive or negative selection, and the exact arrangement of the various nucleic acid segments, as will be readily apparent to those skilled in the art. In some preferred embodiments, the selection scheme results in selection of or enrichment for only one or more desired Products. As defined herein, selecting for a nucleic acid molecule includes (a) selecting or enriching for the presence of the desired nucleic acid molecule, and (b) selecting or enriching against the presence of nucleic acid molecules that are not the desired nucleic acid molecule.

In one embodiment, the selection schemes (which can be carried out in reverse) will take one of three formns, which will be discussed in terms of FIG. 1. The first, exemplified herein with a Selectable marker and a repressor therefore, selects for molecules having segment D and lacking segment C. The second selects against molecules having segment C and for molecules having segment D. Possible embodiments of the second form would have a nucleic acid segment carrying a gene toxic to cells into which the in vitro reaction products are to be introduced. A toxic gene can be a nucleic acid that is expressed as a toxic gene product (a toxic protein or RNA), or can be toxic in and of itself. (In the latter case, the toxic gene is understood to carry its classical definition of “heritable trait”.)

Examples of such toxic gene products are well known in the art, and include, but are not limited to, restriction endonucleases (e.g., DpnI), apoptosis-related genes (e.g. ASK1 or members of the bcl-2/ced-9 family), retroviral genes including those of the human immunodeficiency virus (HTV), defensins such as NP-1, inverted repeats or paired palindromic nucleic acid sequences, bacteriophage lytic genes such as those from (DX174 or bacteriophage T4; antibiotic sensitivity genes such as rpsL, antimicrobial sensitivity genes such as pheS, plasmid killer genes, eukaryotic transcriptional vector genes that produce a gene product toxic to bacteria, such as GATA-1, and genes that kill hosts in the absence of a suppressing function, e.g., kicB, ccdB, ΦX174 E (Liu, Q. et al., Curr. Biol. 8:1300-1309 (1998)), and other genes that negatively affect replicon stability and/or replication. A toxic gene can alternatively be selectable in vitro, e.g., a restriction site.

Many genes coding for restriction endonucleases operably linked to inducible promoters are known, and may be used in the present invention.

See, e.g. U.S. Pat. Nos. 4,960,707 (DpnI and DpnII); 5,000,333, 5,082,784 and 5,192,675 (KpnI); 5,147,800 (NgoAIII and NgoAI); 5,179,015 (FspI and HaeIII): 5,200,333 (HaeII and TaqI); 5,248,605 (HpaII); 5,312,746 (ClaI); 5,231,021 and 5,304,480 (XhoI and XhoII); 5,334,526 (AluI); 5,470,740 (NsiI); 5,534,428 (SstIISacI); 5,202,248 (NcoI); 5,139,942 (NdeI); and 5,098,839 (PacI). See also Wilson, G. G., Nucl. Acids Res. 19:2539-2566 (1991); and Lunnen, K. D., et al., Gene 74:25-32 (1988).

In the second form, segment D carries a Selectable marker. The toxic gene would eliminate transformants harboring the Vector Donor, Cointegrate, and Byproduct molecules, while the Selectable marker can be used to select for cells containing the Product and against cells harboring only the Insert Donor.

The third form selects for cells that have both segments A and D in cis on the same molecule, but not for cells that have both segments in trans on different molecules. This could be embodied by a Selectable marker that is split into two inactive fragments, one each on segments A and D.

The fragments are so arranged relative to the recombination sites that when the segments are brought together by the recombination event, they reconstitute a functional Selectable marker. For example, the recombinational event can link a promoter with a structural nucleic acid molecule (e.g., a gene), can link two fragments of a structural nucleic acid molecule, or can link nucleic acid molecules that encode a heterodimeric gene product needed for survival, or can link portions of a replicon.

Site-specific recombinase: As used herein, a site specific recombinase is a type of recombinase which typically has at least the following four activities (or combinations thereof): (1) recognition of one or two specific nucleic acid sequences; (2) cleavage of said sequence or sequences; (3) topoisomerase activity involved in strand exchange; and (4) ligase activity to reseal the cleaved strands of nucleic acid. See Sauer, B., Current Opinions in Biotechnology 5:521-527 (1994). Conservative site-specific recombination is distinguished from homologous recombination and transposition by a high degree of specificity for both partners. The strand exchange mechanism involves the cleavage and rejoining of specific nucleic acid sequences in the absence of DNA synthesis (Landy, A. (1989) Ann. Rev. Biochem. 58:913-949).

Vector: As used herein, a vector is a nucleic acid molecule (preferably DNA) that provides a useful biological or biochemical property to an Insert. Examples include plasmids, phages, autonomously replicating sequences (ARS), centromeres, and other sequences which are able to replicate or be replicated in vitro or in a host cell, or to convey a desired nucleic acid segment to a desired location within a host cell. A Vector can have one or more restriction endonuclease recognition sites at which the sequences can be cut in a determinable fashion without loss of an essential biological function of the vector, and into which a nucleic acid fragment can be spliced in order to bring about its replication and cloning. Vectors can further provide primer sites, e.g., for PCR, transcriptional and/or translational initiation and/or regulation sites, recombinational signals, replicons, Selectable markers, etc. Clearly, methods of inserting a desired nucleic acid fragment which do not require the use of recombination, transpositions or restriction enzymes (such as, but not limited to, UDG cloning of PCR fragments (U.S. Pat. No. 5,334,575, entirely incorporated herein by reference), TA Cloning® brand PCR cloning (Invitrogen Corporation, Carlsbad, Calif.) (also known as direct ligation cloning), and the like) can also be applied to clone a fragment into a cloning vector to be used according to the present invention. The cloning vector can further contain one or more selectable markers suitable for use in the identification of cells transformed with the cloning vector.

Subcloning vector: As used herein, a subcloning vector is a cloning vector comprising a circular or linear nucleic acid molecule which includes preferably an appropriate replicon. In the present invention, the subcloning vector (segment D in FIG. 1) can also contain functional and/or regulatory elements that are desired to be incorporated into the final product to act upon or with the cloned nucleic acid Insert (segment A in FIG. 1). The subcloning vector can also contain a Selectable marker (preferably DNA).

Vector Donor: As used herein, a Vector Donor is one of the two parental nucleic acid molecules (e.g. RNA or DNA) of the present invention which carries the nucleic acid segments comprising the nucleic acid vector which is to become part of the desired Product. The Vector Donor comprises a subcloning vector D (or it can be called the cloning vector if the Insert Donor does not already contain a cloning vector) and a segment C flanked by recombination sites (see FIG. 1). Segments C and/or D can contain elements that contribute to selection for the desired Product daughter molecule, as described above for selection schemes. The recombination signals can be the same or different, and can be acted upon by the same or different recombinases. In addition, the Vector Donor can be linear or circular.

Primer: As used herein, a primer is a single stranded or double stranded oligonucleotide that is extended by covalent bonding of nucleotide monomers during amplification or polymerization of a nucleic acid molecule (e.g. a DNA molecule). In one aspect, the primer may be a sequencing primer (for example, a universal sequencing primer). In another aspect, the primer may comprise a recognition site (e.g., a recombination site, topoisomerase site, etc.) or portion thereof.

Template: As used herein, a template is a double stranded or single stranded nucleic acid molecule which is to be amplified, synthesized or sequenced. In the case of a double-stranded DNA molecule, denaturation of its strands to form a first and a second strand is preferably performed before these molecules may be amplified, synthesized or sequenced, or the double stranded molecule may be used directly as a template. For single stranded templates, a primer complementary to at least a portion of the template is hybridized under appropriate conditions and one or more polypeptides having polymerase activity (e.g. DNA polymerases and/or reverse transcriptases) may then synthesize a molecule complementary to all or a portion of the template. Alternatively, for double stranded templates, one or more transcriptional regulatory sequences (e.g., one or more promoters) may be used in combination with one or more polymerases to make nucleic acid molecules complementary to all or a portion of the template. The newly synthesized molecule, according to the invention, may be of equal or shorter length compared to the original template. Mismatch incorporation or strand slippage during the synthesis or extension of the newly synthesized molecule may result in one or a number of mismatched base pairs. Thus, the synthesized molecule need not be exactly complementary to the template. Additionally, a population of nucleic acid templates may be used during synthesis or amplification to produce a population of nucleic acid molecules typically representative of the original template population.

Incorporating: As used herein, incorporating means becoming a part of a nucleic acid (e.g., DNA) molecule or primer.

Library: As used herein, a library is a collection of nucleic acid molecules (circular or linear). In one: embodiment, a library may comprise a plurality (i.e., two or more) of nucleic acid molecules, which may or may not be from a common source organism, organ, tissue, or cell. In another embodiment, a library is representative of all or a portion or a significant portion of the nucleic acid content of an organism (a “genomic” library), or a set of nucleic acid molecules representative of all or a portion or a significant portion of the expressed nucleic acid molecules (a cDNA library or segments derived therefrom) in a cell, tissue, organ or organism. A library may also comprise random sequences made by de novo synthesis, mutagenesis of one or more sequences and the like. Such libraries may or may not be contained in one or more vectors.

Amplification: As used herein, amplification is any in vitro method for increasing a number of copies of a nucleotide sequence with the use of one or more polypeptides having polymerase activity (e.g., one or more nucleic acid polymerases or one or more reverse transcriptases). Nucleic acid amplification results in the incorporation of nucleotides into a DNA and/or RNA molecule or primer thereby forming a new nucleic acid molecule complementary to a template. The formed nucleic acid molecule and its template can be used as templates to synthesize additional nucleic acid molecules. As used herein, one amplification reaction may consist of many rounds of nucleic acid replication. DNA amplification reactions include, for example, polymerase chain reaction (PCR). One PCR reaction may consist of 5 to 100 cycles of denaturation and synthesis of a DNA molecule.

Nucleotide: As used herein, a nucleotide is a base-sugar-phosphate combination. Nucleotides are monomeric units of a nucleic acid molecule (DNA and RNA). The term nucleotide includes ribonucleoside triphosphates ATP, UTP, CTG, GTP and deoxyribonucleoside triphosphates such as dATP, dCTP, dITP, dUTP, dGTP, dTTP, or derivatives thereof. Such derivatives include, for example, [S]dATP, 7-deaza-dGTP and 7-deaza-dATP. The term nucleotide as used herein also refers to dideoxyribonucleoside triphosphates (ddNTPs) and their derivatives. Illustratative examples of dideoxyribonucleoside triphosphates include, but are not limited to, ddATP, ddCTP, ddGTP, ddITP, and ddTTP. According to the present invention, a “nucleotide” may be unlabeled or detectably labeled by well known techniques. Detectable labels include, for example, radioactive isotopes, fluorescent labels, chemiluminescent labels, bioluminescent labels and enzyme labels.

Nucleic acid molecule: As used herein, a nucleic acid molecule is a sequence of contiguous nucleotides (riboNTPs, dNTPs or ddNTPs, or combinations thereof) of any length, which may encode a full-length polypeptide or a fragment of any length thereof, or which may be non-coding. As used herein, the terms “nucleic acid molecule” and “polynucleotide” may be used interchangeably.

Oligonucleotide: As used herein, an oligonucleotide is a synthetic or natural molecule comprising a covalently linked sequence of nucleotides.

Nucleotides may be joined by a phosphodiester bond between the 3′ position of the pentose of one nucleotide and the 5′ position of the pentose of the adjacent nucleotide or other types of bonds (e.g., peptide bonds (PNA) known to those skilled in the art.

siRNA: As used herein small interfering RNA of siRNA refers to double-stranded RNA (dsRNA) molecules. Such molecules have been used to modulate protein expression levels by inducing the degradation of mRNA. See, for example, Fire, et al., Nature 391:806-811 (1998), Tuschl, et al., Genes Dev., 13:3191-3197 (1999), Elbashir, et al., Genes Dev., 15:188-200 (2001), and Elbashir, et al., Nature, 411:494-498 (2001). Such molecules may be from about 15 to about 50 nucleotides in length and may contain 5′ and/or 3′-overhanging sequences.

Polypeptide: As used herein, a polypeptide is a sequence of contiguous amino acids, of any length. As used herein, the terms “peptide,” “oligopeptide,” or “protein” may be used interchangeably with the term “polypeptide.”

Hybridization: As used herein, the terms hybridization and hybridizing refer to base pairing of two complementary single-stranded nucleic acid molecules (RNA and/or DNA) to give a double stranded molecule. As used herein, two nucleic acid molecules may be hybridized, although the base pairing is not completely complementary. Accordingly, mismatched bases do not prevent hybridization of two nucleic acid molecules provided that appropriate conditions, well known in the art, are used. In some aspects, hybridization is said to be under “stringent conditions.” By “stringent conditions” as used herein is meant overnight incubation at 42° C. in a solution comprising: 50% formamide, 5×SSC (150 mM NaCl, 15 mM trisodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65° C.

Other terms used in the fields of recombinant nucleic acid technology and molecular and cell biology as used herein will be generally understood by one of ordinary skill in the applicable arts.

Overview

The present invention provides, in part, materials and methods that may be used to clone and/or mutagenize a nucleic acid of interest. In some embodiments, a nucleic acid of interest (e.g., a cDNA) may be cloned and a mutation introduced into the cloned nucleic acid (e.g., a point mutation). In some embodiments, the mutation may be to remove a stop codon normally present in the cDNA sequence. The process of cloning may entail the addition of adapter sequences to the sequence of interest. Adapter sequences may facilitate the cloning of the sequence of interest.

In some embodiments, the present invention relates to a method for the preparation of nucleic acid molecules having recombination sites flanking a sequence of interest. In a preferred embodiment, the nucleic acid molecules will be of more than a single type. In a preferred embodiment, one type of nucleic acid molecule will comprise a sequence that is identical to all or a portion of a template nucleic acid molecule. Another type of nucleic acid molecule produced using the methods of the present invention will comprise a sequence that differs from all or a portion of a template nucleic acid molecule in that one or more nucleotides have been changed as compared to the corresponding sequence in the template or portion thereof. In some embodiments, the nucleotide change or changes may remove a stop codon from the sequence of interest.

In some embodiments, the present invention relates to a unique method for the selective and efficient preparation of a GATEWAY™ entry clone of a native-type cDNA and a C-terminal fused-type cDNA by means of a polymerase chain reaction (PCR). In a particular embodiment, the present invention uses a two-step adapter PCR for constructing PCR products of cDNAs, each having an adapter with a recombination sequence (e.g., attB1) at the 5′-terminus and a recombination sequence, preferably a different recombination sequence (e.g., attB2), at the 3′-terminus, which each acts as a precursor for the preparation of the GATEWAY™ entry clone of the native-type cDNA and the C-terminal fused-type cDNA from DNA fragments containing an objective genomic cDNA.

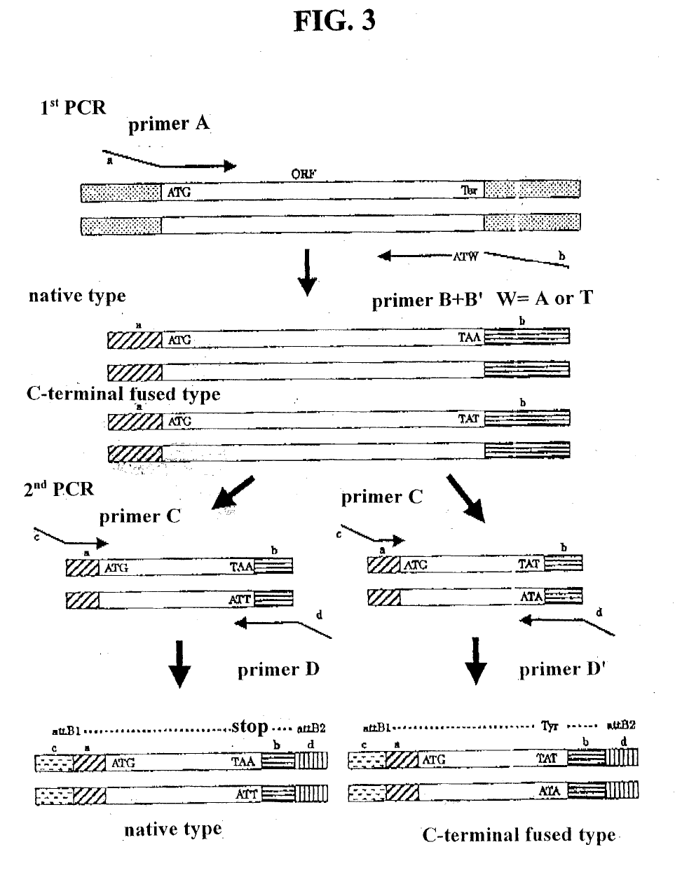

The two-step adapter PCR method according to the present invention comprises a first PCR step and a second PCR step, the first PCR step being for amplifying the desired sequence from the template nucleic acid molecule (e.g., cDNA) by using the 5′-terminal primer A with the adapter a and a mixture of the 3′-terminal primer B and the 3′-terminal primer B′ each containing the adapter b and having a different sequence of bases at a site hybridizable with a stop codon of the template, and the second PCR step being for amplifying the amplified product obtained in the first PCR step as a template by using the 5′-terminal common primer C having the adapter c with the sequence attB1 acting as the site connectable to a plasmid at the 5′-terminus of the PCR product and the native-type common primer D or the C-terminal fused-type common primer D′, each having the adapter d containing the sequence attB2 acting as the site connectable to the plasmid, as a 3′-terminal PCR primer, thereby efficiently yielding a PCR product of the native-type cDNA and the C-terminal fused-type cDNA, each having the sequence attB1 at the 5′-terminus and the sequence attB2 at the 3′-terminus. A schematic illustration of this embodiment is shown in FIG. 3.

Primers of the Invention

Methods of the invention may entail the use of one or more primers in an amplification reaction. Primers of the invention may be of any length and typically contain one or more sequences complementary to one or more sequences of the nucleic acid template to be amplified. The length of a sequence on the primer complementary to a sequence of the template may be varied. Typically, a primer may contain a sequence of from about 5 to about 100, from about 5 to about 75, from about 5 to about 50, from about 5 to about 40, from about 5 to about 30, from about 5 to about 25, from about 5 to about 20, from about 5 to about 15, from about 5 to about 10, from about 10 to about 100, from about 10 to about 75, from about 10 to about 50, from about 10 to about 40, from about 10 to about 30, from about 10 to about 25, from about 10 to about 20, or from about 10 to about 15 nucleotides in length that is complementary to a sequence of the template. In some embodiments, primers of the invention may comprise sequences exactly complementary to the sequence of interest that are located at the 3′-end of the primer.

Primers of the invention may comprise a sequence that is incompletely complementary to a sequence on the template nucleic acid (i.e., contains mismatched nucleotides relative to the template) in the portion of the primer intended to anneal to the template. For example, a primer may contain a sequence that has one or more bases (e.g., two, three, four, five, six, seven, eight, nine, ten, etc.) that do not base pair with the template when the primer is annealed to the template. Extension of such a primer and subsequent amplification results in the production of a PCR product that contains one or more mutations relative to the sequence of the original template nucleic acid molecule. Mis-matches may be located anywhere in the sequence of the primer complementary to the template. In some embodiments, a primer of the invention has at least one mis-matched nucleotide located at or near the 3′-end of the primer. A mis-matched nucleotide may be located about 30 nucleotides, about 25 nucleotides, about 20 nucleotides, about 15 nucleotides, about 10 nucleotides, about 5 nucleotides, about 4 nucleotides, about 3 nucleotides, about 2 nucleotides, about 1 nucleotide (i.e., adjacent to) or at the 3′-most nucleotide of the primer. In one embodiment, a primer of the invention may contain a single mis-matched nucleotide and the mis-matched nucleotide may be located at the 3′-most nucleotide.

A primer may contain one or more nucleotides that are mis-matched relative to the sequence of a template nucleic acid molecule. The mis-matched nucleotides may be distributed throughout the primer sequence or may be adjacent each other or combinations thereof. In primers that contain one or more mis-matches, the portion of the primer intended to anneal to the template molecule may be from about 50% to about 99%, from about 60% to about 99%, from about 70% to about 99%, from about 80% to about 99%, from about 90% to about 99%, or from about 95% to about 99% exactly complementary to the template over the length of the portion of the primer intended to anneal to the template.

Primers of the invention containing mis-matches may anneal to a template under various conditions. The selection of an appropriate annealing temperature for a primer containing one or more mismatches is a routine task for one of ordinary skill in the art. An annealing temperature may be from about 3° C. to about 20° C., from about 5° C. to about 20° C., from about 10° C. to about 20° C., or from about 15° C. to about 20° C. below the predicted Tₘ of the template-primer duplex. Tₘ may be predicted using methods well known in the art, for example, using the formula

Tₘ=81.5−16.6(log₁₀[Na⁺])+0.41(% G+C)−(600/N)

where N=length of the portion of the primer that anneals to the template. Those skilled in the art will appreciate that there is a reduction in Tₘ of approximately 1-1.5° C. for every 1% of mismatching of bases. See Sambrook, et al. Molecular Cloning: A Laboratory Manual 2^{nd} Edition, 1989, Chapter 11, pp.11.46-11.47, Cold Spring Harbor Laboratory Press Cold Spring Harbor, NY.

Primers of the invention may contain one or more adapter sequences, which may be of any length. An adapter sequence may not be complementary to a sequence on the template nucleic acid. An adapter sequence may be any sequence. For example, an adapter sequence may provide a site at which a primer used in a subsequent reaction (e.g., a PCR reaction) may anneal. An adapter sequence may contain all or a portion of one or more of a recombination site sequence, a topoisomerase recognition site sequence, a restriction enzyme recognition site sequences and combinations thereof. In some embodiments, an adapter sequence may comprise all or a portion of one or more recombination site sequences (e.g., att sequences, lox, sequences, etc.). In some embodiments, a primer of the invention may comprise all or a portion of a recombination site sequence selected from the group consisting of an attB, attP, attL, attR, attB1, attP1, attL1, attR1, attB2, attP2, attL2, attR2, attB5, attP5, attL5, attR5, attB11, attP11, attL11, attR11, attB17, attP17, attL17, attR17, attB19, attP19, attL19, attR19, attB20, attP20, attL20, attR20, attB21, attP21, attL21, and attR21.

In some embodiments, adapter sequences may comprise sequences encoding one or more amino acids. For example, an adapter sequence may encode one or more peptide tags. Suitable tags include, but are not limited to metal binding domains (e.g., polyhisitidine), epitopes (e.g., V5 epitope) and other sequences known in the art. Such tag-encoding sequences may be introduced on the N- and/or C-terminal of the PCR products produced by methods of the present invention. In some embodiments, an adapter sequence may encode a tag sequence and may also encode a cleavage site sequence, for example, between the tag and a polypeptide encoded by the template nucleic acid molecule. After transcription and translation of the tag-containing polypeptide, all or a portion of the tag sequence may be removed by cleavage with a protease enzyme that recognizes the cleavage site. Examples of suitable cleavage sites include, but are not limited to, the Factor Xa cleavage site having the sequence Ile-Glu-Gly-Arg (SEQ ID NO: 13), which is recognized and cleaved by blood coagulation factor Xa, and the thrombin cleavage site having the sequence Leu-Val-Pro-Arg (SEQ ID NO: 14), which is recognized and cleaved by thrombin. Other suitable cleavage sites are known to those skilled in the art and may be used in conjunction with the present invention.

Adapter sequences of the primers of the present invention may comprise other functional sequences that may be desirable in the amplified product. Such functional sequences may be incorporated on either or both ends of the nucleic acid molecules produced by methods of the invention. When an mRNA is transcribed from a nucleic acid molecule made according to the present invention, the mRNA may have functional sequences at 5′ and/or 3′ end of a coding sequence of the mRNA. When a polypeptide is translated from such an mRNA, the polypeptide may have peptide regions encoded by such functional sequences at the N and/or C-terminus of the polypeptide.

In some embodiments, it may be desirable to incorporate one or more regulatory sequences (e.g., promoter, repressor, enhancer, etc.), sequences that control translation (e.g., Shine-Dalgarno, Kozac, etc.) and the like. Suitable promoter sequences that may be incorporated include, but are not limited to, constitutive or regulatable (i.e., inducible or derepressible) promoters. Examples of constitutive promoters include the int promoter of bacteriophage λ, and the bla promoter of the β-lactamase gene of pBR322. Examples of inducible prokaryotic promoters include the major right and left promoters of bacteriophage λ (P_{R} and P_{L}), trp, recA, lacZ, lacI, tet, gal, trc, ara BAD (Guzman, et al., 1995, J. Bacteriol. 177(14):4121-4130) and tac promoters of E. coli. The B. subtilis promoters include α-amylase (Ulmanen et al., J. Bacteriol 162:176-182 (1985)) and Bacillus bacteriophage promoters (Gryczan, T., In: The Molecular Biology Of Bacilli, Academic Press, New York (1982)). Streptomyces promoters are described by Ward et al., Mol. Gen. Genet. 203:468478 (1986)). Prokaryotic promoters are also reviewed by Glick, J. Ind. Microbiol. 1:277-282 (1987); Cenatiempto, Y., Biochimie 68:505-516 (1986); and Gottesman, Ann. Rev. Genet. 18:415-442 (1984). Expression in a prokaryotic cell also requires the presence of a ribosomal binding site upstream of the gene-encoding sequence. Such ribosomal binding sites are disclosed, for example, by Gold et al., Ann. Rev. Microbiol. 35:365404 (1981).

When promoters are incorporated into nucleic acid molecules produced by methods of the present invention, they may be incorporated at either end of either or both strands of a double-stranded nucleic acid molecule.

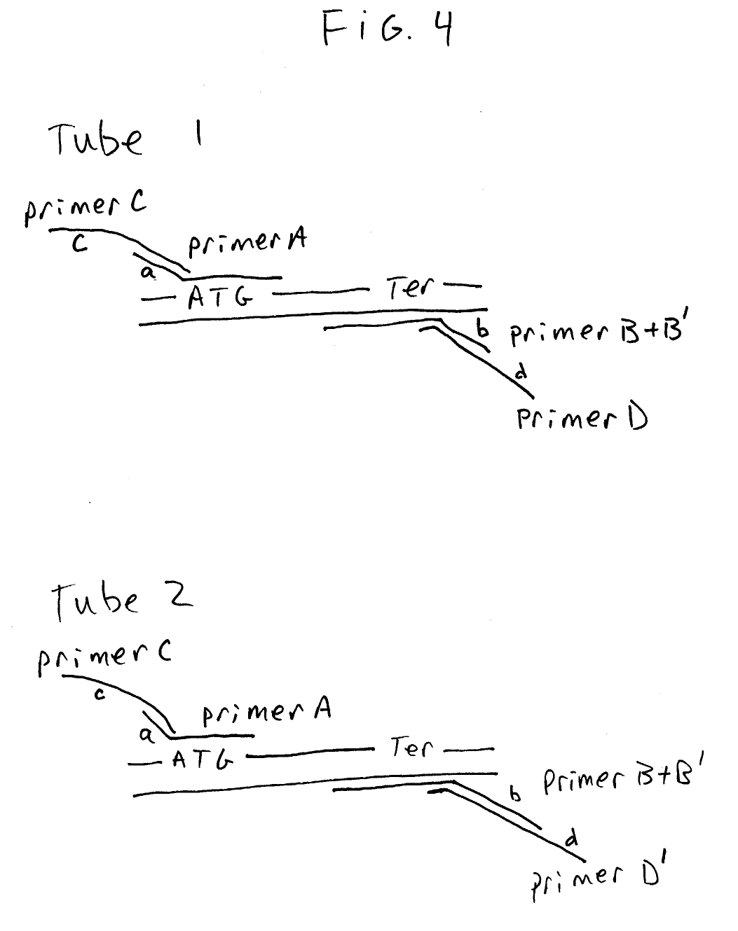

In some embodiments, primers B and/or B′ (FIGS. 3 and 4) may comprise a sequence that exactly hybridizes to a sequence of interest on a template nucleic acid molecule. Such a sequence may be from about 10 to about 30, from about 10 to about 25, from about 10 to about 20, from about 10 to about 19, from about 10 to about 18, from about 10 to about 17, from about 10 to about 16, from about 10 to about 15, from about 10 to about 14, from about 10 to about 13, from about 10 to about 12, or from about 10 to about 11 in length. In some embodiments, an exactly hybridizing sequence may be located at the 3′-end of the primers B and/or B′.

In some embodiments, primers B and/or B′ may have two nucleotides that may be mis-matched with regard to the last two nucleotides of a stop codon present in the sequence of interest. For example, primer B may have a sequence 3′-TT-5′ that aligns with the last two nucleotides of a stop codon present in the sequence of interest. Amplification with such a primer will result in changing the sequence of the stop codon to be a TAA stop codon. Likewise, primer B′ may have a sequence 3′-TA-5′ that aligns with the last two nucleotides of a stop codon present in the sequence of interest. Amplification with such a primer will result in changing a stop codon to a TAT codon. Thus, in some embodiments, methods of the present invention may be used to convert all stop codons in a population of nucleic acid molecules into TAA stop codons.

In some embodiments, primers B and/or B′ may have the following arrangement of sequences: 5′-adapter b-sequence aligning with stop codon-sequence complementary to sequence of interest-3′.

In some embodiments, primes D and/or D′ (FIGS. 3 and 4) may comprise a sequence that anneals to PCR products comprising adapter sequence b and may also comprise an adapter sequence d. In some embodiments, primers D and/or D′ may anneal to the PCR products containing adapter sequence b such that the 3′-most nucleotide of primers D and/or D′ align with a nucleotide corresponding to one of the nucleotides of a stop codon in the sequence of interest. Those skilled in the art will appreciate that, as a result of being amplified with primers B and/or B′, the PCR product may no longer have a stop codon or may have an altered stop codon at this position. In some embodiments, primer D may have a T at the 3′-terminus and may anneal to PCR products comprising adapter b such that the 3-terminus anneals to the last nucleotide of a TAA stop codon present in the PCR product. In some embodiments, primer D′ may have an A at the 3′-teiminus and may anneal to PCR products comprising adapter b such that the 3-terminus anneals to the last nucleotide of a TAT codon present in the PCR product that corresponds to a stop codon present in the sequence of interest on the template nucleic acid molecule.

The sequences of primers and adapter sequences specifically referred to herein are provided below:

| | |
|---|---|
| GGAGATAGAA CC | (SEQ ID NO:1) |
| GAAAGCTGGG T | (SEQ ID NO:2) |
| ACAAGTTTGT ACAAAAAAGC AGGCT | (SEQ ID NO:3) |
| GGGGACAAGT TTGTACAAAA AAGCAGGCTT | (SEQ ID NO:4) |
| CGAAGGAGAT AGAACC | |
| ACCACTTTGT ACAAGAAAGC TGGGT | (SEQ ID NO:5) |
| GGGGACCACT TTGTACAAGA AAGCTGGGTC | (SEQ ID NO:6) |
| GGGGACCACT TTGTACAAGA AAGCTGGGTC | (SEQ ID NO:7) |
| TTA | |
| GGGGACCACT TTGTACAAGA AAGCTGGGTC | (SEQ ID NO:8) |
| ATA | |
| GGGGACCACT TTGTACAAGA AAGCTGGGTC | (SEQ ID NO:9) |
| TCA | |
| GGGGACCACT TTGTACAAGA AAGCTGGGTC | (SEQ ID NO:10) |
| ACA | |
| GGGGACCACT TTGTACAAGA AAGCTGGGTC | (SEQ ID NO:11) |
| TCC | |
| GGGGACCACT TTGTACAAGA AAGCTGGGT | (SEQ ID NO:12) |

Recombination Sites

Recombination sites for use in the invention may be any nucleic acid sequence that can serve as a substrate in a recombination reaction. Such recombination sites may be wild-type or naturally occurring recombination sites or modified or mutant recombination sites. Examples of recombination sites for use in the invention include, but are not limited to, phage-lambda recombination sites (such as attP, attB, attL, and attR and mutants or derivatives thereof) and recombination sites from other bacteriophage such as phi80, P22, P2, 186, P4 and P1 (including lox sites such as loxP and loxP511). Other suitable recombination proteins and mutant, modified, variant, or derivative recombination sites for use in the invention include those described in U.S. Pat. Nos. 5,888,732, 6,143,557, 6,171,861, 6,270,969, and 6,277,608 and in U.S. application Ser. no. 09/438,358 (filed Nov. 12, 1999), based upon U.S. provisional application no. 60/108,324 (filed Nov. 13, 1998).

Mutating specific residues in the core region of the att site can generate a large number of different att sites. As with the att1 and att2 sites utilized in GATEWAY™ , each additional mutation potentially creates a novel att site with unique specificity that will recombine only with its cognate partner att site bearing the same mutation and will not cross-react with any other mutant or wild-type att site. Mutated att sites (e.g., attB 1-10, attP 1-10, attR 1-10 and attL 1-10) are described in U.S. provisional patent application Nos. 60/122,389, filed Mar. 2, 1999, 60/126,049, filed Mar. 23, 1999, 60/169,983, filed Dec. 10, 1999, and 60/188,000, filed Mar. 9, 2000, and in U.S. application Ser. Nos. 09/517,466, filed Mar. 2, 2000, and 09/732,914, filed Dec. 11, 2000 (published as 20020007051-A1) the disclosures of which are specifically incorporated herein by reference in their entirety. Other suitable recombination sites and proteins are those associated with the GATEWAY™ Cloning Technology available from Invitrogen Corporation, Carlsbad, Calif., and described in the product literature of the GATEWAY™ Cloning Technology, the entire disclosures of all of which are specifically incorporated herein by reference in their entireties.

For example, mutated att sites that may be used in the practice of the present invention include attB1 (AGCCTGCTTT TTTGTACAAA CTTGT (SEQ ID NO: 15)), attP1 (TACAGGTCAC TAATACCATC TAAGTAGTTG ATTCATAGTG ACTGGATATG TTGTGTTTTA CAGTATTATG TAGTCTGTTT TITATGCAAA ATCTAATTTA ATATATTGAT ATTTATATCA TTTTACGTTT CTCGTTCAGC TTTTTTGTAC AAAGTTGGCA TTATAAAAAA GCATTGCTCA TCAATTTGTT GCAACGAACA GGTCACTATC. AGTCAAAATA AAATCATTAT TTG (SEQ ID NO: 16)), attLl (CAAATAATGA TTTTATTTTG ACTGATAGTG ACCTGTTCGT TGCAACAAAT TGATAAGCAA TGCTTTTTTA TAATGCCAAC TTTGTACAAA AAAGCAGGCT (SEQ ID NO: 17)), and attR1 (ACAAGTTTGT ACAAAAAAGC TGAACGAGAA ACGTAAAATG ATATAAATAT CAATATATTA AATTAGATTT TGCATAAAAA ACAGACTACA TAATACTGTA AAACACAACA TATCCAGTCA CTATG (SEQ ID NO: 18)). Table 1 provides the sequences of the regions surrounding the core region for the wild type att sites (attB0, P0, R0, and L0) as well as a variety of other suitable recombination sites. Those skilled in the art will appreciated that the remainder of the site is the same as the corresponding site (B, P, L, or R) listed above.

TABLE 1
Nucleotide sequences of representative att sites.

| | | |
|---|---|---|
| attB0 | AGCCTGCTTT TTTATACTAA CTTGAGC | (SEQ ID NO:19) |
| attP0 | GTTCAGCTTT TTTATACTAA GTTGGCA | (SEQ ID NO:20) |
| attL0 | AGCCTGCTTT TTTATACTAA GTTGGCA | (SEQ ID NO:21) |
| attR0 | GTTCAGCTTT TTTATACTAA CTTGAGC | (SEQ ID NO:22) |
| attB1 | AGCCTGCTTT TTTGTACAAA CTTGT | (SEQ ID NO:23) |
| attP1 | GTTCAGCTTT TTTGTACAAA GTTGGCA | (SEQ ID NO:24) |
| attL1 | AGCCTGCTTT TTTGTACAAA GTTGGCA | (SEQ ID NO:25) |
| attR1 | GTTCAGCTTT TTTGTACAAA CTTGT | (SEQ ID NO:26) |
| attB2 | ACCCAGCTTT CTTGTACAAA GTGGT | (SEQ ID NO:27) |
| attP2 | GTTCAGCTTT CTTGTACAAA GTTGGCA | (SEQ ID NO:28) |
| attL2 | ACCCAGCTTT CTTGTACAAA GTTGGCA | (SEQ ID NO:29) |
| attR2 | GTTCAGCTTT CTTGTACAAA GTGGT | (SEQ ID NO:30) |
| attB5 | CAACTTTTCT ATACAAAGTT GT | (SEQ ID NO:31) |
| attP5 | GTTCAACTTT ATTATACAAA GTTGGCA | (SEQ ID NO:32) |
| attL5 | CAACTTTATT ATACAAAGTT GGCA | (SEQ ID NO:33) |
| attR5 | GTTCAACTTT ATTATACAAA GTTGT | (SEQ ID NO:34) |
| attB11 | CAACTTTTCT ATACAAAGTT GT | (SEQ ID NO:35) |
| attP11 | GTTCAACTTT TCTATACAAA GTTGGCA | (SEQ ID NO:36) |
| attL11 | CAACTTTTCT ATACAAAGTT GGCA | (SEQ ID NO:37) |
| attR11 | GTTCAACTTT TCTATACAAA GTTGT | (SEQ ID NO:38) |
| attB17 | CAACTTTTGT ATACAAAGTT GT | (SEQ ID NO:39) |
| attP17 | GTTCAACTTT TGTATACAAA GTTGGCA | (SEQ ID NO:40) |
| attL17 | CAACTTTTGT ATACAAAGTT GGCA | (SEQ ID NO:41) |
| attR17 | GTTCAACTTT TGTATACAAA GTTGT | (SEQ ID NO:42) |
| attB19 | CAACTTTTTC GTACAAAGTT GT | (SEQ ID NO:43) |
| attP19 | GTTCAACTTT TTCGTACAAA GTTGGCA | (SEQ ID NO:44) |
| attL19 | CAACTTTTTC GTACAAAGTT GGCA | (SEQ ID NO:45) |
| attR19 | GTTCAACTTT TTCGTACAAA GTTGT | (SEQ ID NO:46) |
| attB20 | CAACTTTTTG GTACAAAGTT GT | (SEQ ID NO:47) |
| attP20 | GTTCAACTTT TTGGTACAAA GTTGGCA | (SEQ ID NO:48) |
| attL20 | CAACTTTTTG GTACAAAGTT GGCA | (SEQ ID NO:49) |
| attR20 | GTTCAACTTT TTGGTACAAA GTTGT | (SEQ ID NO:50) |
| attB21 | CAACTTTTTA ATACAAAGTT GT | (SEQ ID NO:51) |
| attP21 | GTTCAACTTT TTAATACAAA GTTGGCA | (SEQ ID NO:52) |
| attL21 | CAACTTTTTA ATACAAAGTT GGCA | (SEQ ID NO:53) |
| attR21 | GTTCAACTTT TTAATACAAA GTTGT | (SEQ ID NO:54) |

Other recombination sites having unique specificity (i.e., a first site will recombine with its corresponding site and will not recombine or not substantially recombine with a second site having a different specificity) may be used to practice the present invention. Examples of suitable recombination sites include, but are not limited to, loxP sites and derivatives such as loxP511 (see U.S. Pat. No. 5,851,808), frt sites and derivatives, dif sites and derivatives, psi sites and derivatives and cer sites and derivatives. Other systems providing recombination sites and recombination proteins for use in the invention include the FLP/FRT system from Saccharomyces cerevisiae, the resolvase family (e.g., γδ, TndX, TnpX, Tn3 resolvase, Hin, Hjc, Gin, SpCCE1, ParA, and Cin), and IS231 and other Bacillus thuringiensis transposable elements. Other suitable recombination systems for use in the present invention include the XerC and XerD recombinases. Other suitable recombination sites may be found in U.S. Pat. No. 5,851,808 issued to Elledge and Liu which is specifically incorporated herein by reference.

Site-Specific Recombinases

Site-specific recombinases are proteins that are present in many organisms (e.g. viruses and bacteria) and have been characterized as having both endonuclease and ligase properties. These recombinases (along with associated proteins in some cases) recognize specific sequences of bases in a nucleic acid molecule and exchange the nucleic acid segments flanking those sequences. The recombinases and associated proteins are collectively referred to as “recombination proteins” (see, e.g., Landy, A., Current Opinion in Biotechnology 3:699-707 (1993)).

Numerous recombination systems from various organisms have been described. See, e.g., Hoess, et al., Nucleic Acids Research 14(6):2287 (1986); Abremski, et al., J. Biol. Chem. 261(1):391 (1986); Campbell, J. Bacteriol. 174(23):7495 (1992); Qian, et al., J. Biol. Chem. 267(11):7794 (1992); Araki, et al., J. Mol. Biol. 225(1):25 (1992); Maeser and Kahnmann, Mol. Gen. Genet. 230:170-176) (1991); Esposito, et al., Nucl. Acids Res. 25(18):3605 (1997). Many of these belong to the integrase family of recombinases (Argos, et al., EMBO J. 5:433-440 (1986); Voziyanov, et al., Nucl. Acids Res. 27:930 (1999)). Perhaps the best studied of these are the Integrase/att system from bacteriophage (Landy, A. Current Opinions in Genetics and Devel. 3:699-707 (1993), Hoess and Abremski (1990) In Nucleic Acids and Molecular Biology, vol. 4. Eds.: Eckstein and Lilley, Berlin-Heidelberg: Springer-Verlag; pp. 90-109), and the FLP/FRT system from the Saccharomyces cerevisiae 2 μ circle plasmid (Broach, et al., Cell 29:227-234 (1982)).

Topoisomerases

Topoisomerases are categorized as type I, including type IA and type IB topoisomerases, which cleave a single strand of a double stranded nucleic acid molecule, and type II topoisomerases (gyrases), which cleave both strands of a nucleic acid molecule. Type IA and IB topoisomerases cleave one strand of a nucleic acid molecule. Cleavage of a nucleic acid molecule by type IA topoisomerases generates a 5′ phosphate and a 3′ hydroxyl at the cleavage site, with the type IA topoisomerase covalently binding to the 5′ terminus of a cleaved strand. In comparison, cleavage of a nucleic acid molecule by type IB topoisomerases generates a 3′ phosphate and a 5′ hydroxyl at the cleavage site, with the type IB topoisomerase covalently binding to the 3′ terminus of a cleaved strand. As disclosed herein, type I and type II topoisomerases, as well as catalytic domains and mutant forms thereof, are useful for the methods of the invention.

Type IA topoisomerases include E. coli topoisomerase I, E. coli topoisomerase III, eukaryotic topoisomerase II, archeal reverse gyrase, yeast topoisomerase III, Drosophila topoisomerase III, human topoisomerase III, Streptococcus pneumoniae topoisomerase III, and the like, including other type IA topoisomerases (see Berger, Biochim. Biophys. Acta 1400:3-18, 1998; DiGate and Marians, J. Biol. Chem. 264:17924-17930, 1989; Kim and Wang, J. Biol. Chem. 267:17178-17185, 1992; Wilson et al., J. Biol. Chem. 275:1533-1540, 2000; Hanai et al., Proc. Natl. Acad. Sci., USA 93:3653-3657, 1996, U.S. Pat. No. 6,277,620, each of which is incorporated herein by reference). E. coli topoisomerase III, which is a type IA topoisomerase that recognizes, binds to and cleaves the sequence 5′-GCAACTT-3′, can be particularly useful in a method of the invention (Zhang et al., J. Biol. Chem. 270:23700-23705, 1995, which is incorporated herein by reference). A homolog, the trae protein of plasmid RP4, has been described by Li et al., J. Biol. Chem. 272:19582-19587 (1997) and can also be used in the practice of the invention. A DNA-protein adduct is formed with the enzyme covalently binding to the 5′-thymidine residue, with cleavage occurring between the two thymidine residues.

Type IB topoisomerases include the nuclear type I topoisomerases present in all eukaryotic cells and those encoded by vaccinia and other cellular poxviruses (see Cheng et al., Cell 92:841-850, 1998, which is incorporated herein by reference). The eukaryotic type IB topoisomerases are exemplified by those expressed in yeast, Drosophila and mammalian cells, including human cells (see Caron and Wang, Adv. Pharmacol. 29B,:271-297, 1994; Gupta et al., Biochim. Biophys. Acta 1262:1-14, 1995, each of which is incorporated herein by reference; see, also, Berger, supra, 1998). Viral type IB topoisomerases are exemplified by those produced by the vertebrate poxviruses (vaccinia, Shope fibroma virus, ORF virus, fowlpox virus, and molluscum contagiosum virus), and the insect poxvirus (Amsacta moorei entomopoxvirus) (see Shuman, Biochim. Biophys. Acta 1400:321-337, 1998; Petersen et al., Virology 230:197-206, 1997; Shuman and Prescott, Proc. Natl. Acad. Sci., USA 84:7478-7482, 1987; Shuman, J. Biol. Chem. 269:32678-32684, 1994; U.S. Pat. No. 5,766,891; PCTJUS95/16099; PCT[US98/12372, each of which is incorporated herein by reference; see, also, Cheng et al., supra, 1998).

Type II topoisomerases include, for example, bacterial gyrase, bacterial DNA topoisomerase IV, eukaryotic DNA topoisomerase II, and T-even phage encoded DNA topoisomerases (Roca and Wang, Cell 71:833-840, 1992; Wang, J. Biol. Chem. 266:6659-6662, 1991, each of which is incorporated herein by reference; Berger, supra, 1998;). Like the type IB topoisomerases, the type II topoisomerases have both cleaving and ligating activities. In addition, like type IB topoisomerase, substrate nucleic acid molecules can be prepared such that the type II topoisomerase can form a covalent linkage to one strand at a cleavage site. For example, calf thymus type II topoisomerase can cleave a substrate nucleic acid molecule containing a 5′ recessed topoisomerase recognition site positioned three nucleotides from the 5′ end, resulting in dissociation of the three nucleotide sequence 5′ to the cleavage site and covalent binding the of the topoisomerase to the 5′ terminus of the nucleic acid molecule (Andersen et al., supra, 1991). Furthermore, upon contacting such a type II topoisomerase charged nucleic acid molecule with a second nucleotide sequence containing a 3′ hydroxyl group, the type II topoisomerase can ligate the sequences together, and then is released from the recombinant nucleic acid molecule. As such, type II topoisomerases also are useful for performing methods of the invention.

Methods of the Invention

Methods of the invention provide for the rapid and efficient amplification of one or more desired nucleic acid sequences present on one or more template nucleic acid molecules such that the amplified product contains one or more adapter sequences. Methods of the invention also provide for the mutation of one or more nucleotides of the desired nucleotide sequences. In some embodiments, the addition of adapters and the mutation of the nucleotides is accomplished in the same method. Methods of the invention may also include cloning the nucleic acid sequences. Adapter sequences may be incorporated on the N and/or C-terminals of PCR products produced by methods of the present invention. Likewise, mutations may be introduced in the N and/or C-terminal portion of the PCR product relative to the template sequence. For example, an ATG start codon may be changed and/or a stop codon maybe changed using the methods of the invnetion.

In many instances, methods of the invention may involve two PCR reactions. In the first reaction of such embodiments, a template nucleic acid molecule comprising a desired nucleic acid sequence (also referred to a nucleic acid sequence of interest) is amplified using three primers. The sequence of interest may be any nucleotide sequence, for example, all or a portion of a gene, all or a portion of an open reading frame (ORF), all or a portion of a sequence coding for a non-translated RNA (e.g., a tRNA, an antisense RNA, an siRNA, a ribozyme, etc), or any other sequence. For the purpose of example, one embodiment of the methods of the invention will be described below in terms of an ORF, those skilled in the art will readily appreciate that the methods can be used to clone other sequences of interest.

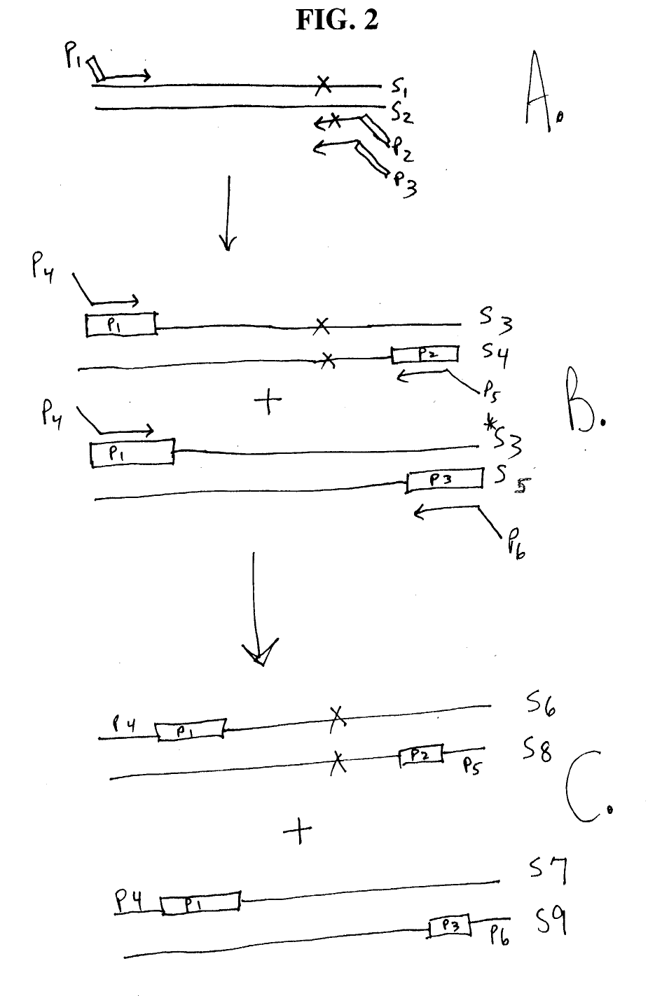

With reference to FIG. 2A, a double-stranded DNA molecule (strands S1 and S2) containing a sequence of interest is contacted with three primers (P1, P2, and P3). One primer anneals to the template at a site flanking the first end of the sequence of interest (P1) and the other two primers anneal to the template at a site flanking the second end of the sequence of interest (P2 and P3). It may be desired to change a nucleotide in the sequence of interest (designated X) at the same time as the sequence is cloned. Methods of the invention are particularly suited for the case where it is desirable to clone both the wildtype sequence of interest and a mutated sequence of interest. In this case, one of the primers that anneal to the second end may contain a sequence complementary to the site to be mutated (P2 containing complement of X) and the other may contain a sequence that has a mismatched base at the nucleotide in question.

After amplification, two different PCR products are obtained. One product containing the sequence of the first and second primer (P1 and P2, strands S3 and S4) and the other containing the sequence of the first and third primer (P1 and P3, strands *S3 and S5). In addition to containing the sequences of the different primers P2 and P3, strand S3 differs from strand *S3 at the position of nucleotide X. As shown in FIG. 2B, strand S3 contains the wildtype nucleotide X and *S3 has a different nucleotide at this position. By including two additional primers (not shown) it is possible to generate four different PCR products that have all the possible nucleotides at the position X.

In some embodiments, primers P2 and P3 may have the same nucleotide sequence except for the nucleotide at position X. In other embodiments, the sequences may be different. For example, the portions of the primers indicated as not annealing to the template in FIG. 2A may be the same or different for primers P2 and P3.

In FIG. 2B, the products of the first PCR reaction are used as templates in a second PCR reaction. The second PCR reaction may be performed in two tubes. In one tube, primers P4 and P5 are used to amplify the product containing the wild type nucleotide at position X while in the other tube primers P4 and P6 are used to amplify the product containing the mutated nucleotide at position X. The products from the second PCR reaction can be cloned using one or another of the techniques described herein. For example, in the case where primer P4 contains an attB1 sequence, primers P5 and P6 may contain an attB2 sequence. After the second PCR reaction, the products may be cloned into a vector containing attP1 and attP2 sites. The primers P4, P5, and P6 may contain recombination site sequences, topoisomerase site sequences, restriction enzyme recognition sequences or combinations thereof. It is not necessary that all the primers have the same type of sequence, for example, primer P4 may have a recombination site sequence while primers P5 and/or P6 may contain a topoisomerase site sequence.

In another embodiment, the second PCR reaction may be performed in a single tube. The reaction product from the first PCR reaction may be contacted with three primers (e.g., P4, P5, and P6). In embodiments of this type, the sequences of P5 and P6 that do not anneal to the template may be different. For example, primer P4 may contain an attB1 recognition sequence, primer P5 may contain an attB2 recognition sequence and primer P6 may contain an attB5 recognition sequence. After the second PCR reaction, a recombination reaction may be conducted with the product and two different vectors, one vector having attP1 and attP2 recombination sites, the other having attP1 and attP5 recombination sites. Preferably the two vectors may be selected for using different selection schemes, for example, each vector may confer a different antibiotic resistance. After transforming the recombination reaction product into a competent host cell, the cells can be split and plated on two different antibiotics to select for the desired clones. Although described above in terms of attB recombination sites, other recombination sites (e.g., loxP sites), topoisomerase sites and/or restriction enzyme sites or combinations thereof may be incorporated into one or more adapters and used to construct nucleic acid molecules according to methods of the present invention. Adjusting the reaction conditions to insert the second PCR products into the vectors (e.g., selecting appropriate polypeptides, buffers, cofactors, etc.) is within the skill of the ordinary practitioner.

In one specific embodiment, in the first step of the PCR method, two kinds of DNAs having sequences of bases, that is, TAA and TAT, or TGA and TGT, or TGA and GGA, at the 3′-terminus of the ORF present on the cDNA are amplified by using the mixture of the primers B and B′ having base sequences, 3′-ATT-5′ and 3′-ATA-5′ (only this combination being indicated in FIG. 3), or 3′-ACT-5′ and 3′-ACA-5′, or 3′-ACT-5′ and 3′-CCT-5′, respectively, at the sequence of bases hybridizable with the stop codon thereof. Then, the second step of the PCR method is carried out by using the PCR products obtained by the first PCR step as templates and, as a 3′-terminal PCR primer, the native-type common primer D as described in SEQ ID NO. 7 or 9 having the sequences 3′-ATT-5′ or 3′-ACT-5′, respectively, at the 3′-terminus or the C-terminal fused-type common primer D′ as described in SEQ ID NO. 8, 10 or 11 having the sequence 3′-ATA-5′, 3′-ACA-5′ or 3′-CCT-5′ at the 3′-terminus of the primer, whereby the native-type cDNA terminating the reading with the stop codon and the C-terminal fused-type cDNA with the stop codon broken and the reading of the C-terminus connected can be selectively and efficiently formed, respectively.

Further, the second step of the PCR method can be conducted by using the 5′-terminal common primer C containing the adapter c and two kinds of the native-type common primer D and the C-terminal fused-type common primer D′, each containing the adapter d, as the 3′-terminal PCR primers, thereby yielding the native-type cDNA and the C-terminal fused-type cDNA, each having the sequence attB1 at the 5′-terminus and the sequence attB2 at the 3′-terminus, respectively, as PCR products. The GATEWAY™ entry clone can then be formed by integrating the resulting cDNAs into the attP plasmid and then introducing them into a competent cell.

The sequence of bases of the adapter a in the 5′-terminal primer A to be used in the first step of the PCR method according to the present invention may be the same as the sequence of bases as described in SEQ ID NO. 1. The sequence of bases of the adapter b in the 3′-terminal primer B and the 3′-terminal primer B′ may be the same as the sequence of bases as described in SEQ ID NO. 2.

Although the 5′-terminal common primer C containing the adapter c is used in the second step of the PCR method according to the present invention, the sequence of bases of the adapter c in the common primer C may be the same as the sequence of bases as described in SEQ ID NO. 3. Further, the sequence of bases of the primer C may be the same as the sequence of bases as described in SEQ ID NO. 4.

On the other hand, although two kinds of the native-type common primer D and the C-terminal fused-type common primer D′, each having the adapter d, are used at the 3′-terminus, the sequence of bases of the adapter d in both of the common primers may be the same as the sequence of bases as described in SEQ ID NO. 6.

In the PCR method according to the present invention, there may be used a DNA segment containing a full length cDNA polynucleotide obtainable by reverse transcription of an RNA. The DNA segment may be derived from natural sources including but being not limited to viruses, yeasts, molds, plants, insects and human beings. Further, it may be prepared from any RNA materials.

In the steps of the PCR method according to the present invention, a variety of DNA polymerases may be used. Preferred ones may include but be not limited to thermally stable DNA polymerases obtainable from a variety of bacteria including but being not limited to Thermus aquaticus (Taq), Thermus thermophilus (Tth), and Pyrococcus furiosus) (Pfu).

Further, in the steps of the PCR method according to the present invention, the reaction efficiency can be improved by using an enhancer solution (Invitrogen Corporation, Carlsbad, Calif. catalog # 11495017). If such an enhancer solution is to be added, the rate of the enhancer solution may amount to from 5% to 15%, preferably from 8% to 12%, with respect to the composition of the PCR reaction mixture.

Moreover, a number of cycles of the reaction program of PCR in the first step of the PCR method according to the present invention may be in the range of preferably from 3 to 10, more preferably from 4 to 6.

A number of cycles of the reaction program of PCR in the second step thereof may be in the range of preferably from 5 to 20, more preferably from 8 to 12.

As an example of the reaction conditions for the first step of the PCR method according to the present invention, the composition of the reaction mixture is illustrated in Table 2 below and the reaction program is illustrated in Table 3 below.

TABLE 2
Composition of Reaction Mixture

| Reagents | Amounts | |
|---|---|---|
| 10x PCR buffer | 2.5 | μl |
| Enhancer solution | 2.5 | μl |
| 10 mM dNTP mix | 0.5 | μl |
| 50 mM MgSO₄ | 1.0 | μl |
| Primer A (10 μM) | 0.5 | μl |
| Primers B and B′ (10 μM) | 0.5 | μl |
| Template DNA | 50 | ng |
| PLATINUM Taq DNA polymerase High Fidelity | 0.1 | μl |
| (5 units/μl) | | |
| Total | 25.0 | μl |

TABLE 3
PCR reaction program

| | | |
|---|---|---|
| 95° C. | 2 | minutes |

followed by 5 cycles under the following conditions

| | | |
|---|---|---|
| 94° C. | 15 | seconds, |
| 55° C. | 30 | seconds, |
| 68° C. | 3 | minutes, and |
| 68° C. | 5 | minutes |

| | |
|---|---|
| 4° C. | To store |

As an example of the reaction conditions for the second step of the PCR method according to the present invention, the composition of the reaction mixture is illustrated in Table 4 below and the reaction program is illustrated in Table 5 below.

TABLE 4
Composition of Reaction Mixture

| Reagents | Amounts | |
|---|---|---|
| 10x PCR buffer | 2.5 | μl |
| Enhancer solution | 2.5 | μl |
| 10 mM dNTP mix | 0.5 | μl |
| 50 mM MgSO₄ | 1.0 | μl |
| Primer C (100 μM) | 0.2 | μl |
| Primer D or D′ (100 μM) | 0.2 | μl |
| Reaction Mixture of First PCR Step | 5.0 | μl |
| SDW | 13.0 | μl |
| PLATINUM Taq polymerase High Fidelity | 0.1 | μl |
| (5 units/μl) | | |
| Total | 25.0 | μl |

TABLE 5
PCR reaction program

| | | |
|---|---|---|
| 95° C. | 2 | minutes |

followed by 3 cycles under the following conditions

| | | |
|---|---|---|
| 94° C. | 15 | seconds, |
| 45° C. | 30 | seconds, |
| 68° C. | 3 | minutes, |

followed by 7 cycles under the following conditions

| | | |
|---|---|---|
| 94° C. | 15 | seconds, |
| 55° C. | 30 | seconds, |
| 68° C. | 3 | minutes, and |
| 68° C. | 5 | minutes |

| | |
|---|---|
| 4° C. | To store |

The reaction conditions for the PCR method indicated in Tables 2 through 5 above, inclusive, are illustrated simply as examples, so that the conditions including but being not limited to the amount of the template DNA and so on may be appropriately selected in accordance with the length of cDNA or other elements for the formation of the entry clone.

It is to be noted that the two-step adapter PCR method according to the present invention can simultaneously form the native-type cDNA and the C-terminal fused-type cDNA, each having the sequence attB1 at the 5′-terminus and the sequence attB2 at the 5′-terminus with high efficiency. The integration of the PCR product of the second PCR step (hereinafter may be referred to as “the second PCR product”) into the attP plasmid for the preparation of the entry clone may be carried out easily by means of a BP reaction. An example of the composition of a reaction mixture for the BP reaction will be indicated in Table 6 below.

TABLE 6
Composition of BP Reaction Mixture

| Reagents | Amounts | |
|---|---|---|
| 5x BP reaction buffer | 2 | μl |
| Second PCR product | 5 | μl |
| pDONR201 (150 ng/μl) | 1 | μl |
| BP CLONASE Enzyme Mixture | 2 | μl |
| Total | 10 | μl |

The BP reaction may be carried out in a manner as will be described hereinafter. A reaction mixture containing no second PCR product was prepared by mixing the raw materials on ice and the second PCR product was added thereto in a predetermined amount, followed by incubating the resultant mixture at 25° C. for 1 hour or more and terminating the reaction by adding 1 μl of 10×proteinase K (2 μg/μl) and incubating the resulting mixture at 37° C. for 10 minutes.

In accordance with the present invention, the GATEWAY™ entry clone may be prepared, for example, by transforming the competent cell in an operational manner as will be described hereinafter. The competent cell was transformed by adding 5 μl of the BP reaction product to 50 μl of a solution of the competent cell on ice and the resulting mixture was then incubated intact for 30 minutes. Thereafter, the resulting cell was subjected to heat shock at 42° C. for 30 seconds and immediately transferred onto ice and allowed to stand for 2 minutes. Then, 250 μl of a SOC culture medium was added thereto and the resulting mixture was subjected to shaking culture for 5 hours. A selection medium plate (an LB medium plate containing 50 μg/ml of kanamycin) was inoculated with the cell so cultured in the amount of 100 μl to 150 μl and incubated at 37° C. Four colonies were picked up for each of the clones grown on the plate and then subjected to shaking culture in 0.2 ml of a TB medium containing kanamycin at the rate of 50 μg/ml. Thereafter, the resulting culture medium was added to a 80% glycerol solution and stirred well, followed by sealing and storing at −80° C.

The resulting clones were confirmed if the objective entry clone was formed.

In accordance with the present invention, the confirmation of the formation of the objective entry clone was conducted by amplifying cDNA by the colony PCR method for the colony obtained from the clone and then stored. As an example of the reaction conditions for the colony PCR method, the composition of the reaction mixture is indicated in Table 7 below and the reaction program is indicated in Table 8 below. Further, the sequence of bases as described in SEQ ID NO. 4 was used for the sequence of bases for the primer C and the sequence of bases as described in SEQ ID NO. 12 was used for the sequence of bases of the attB2 primer.

TABLE 7
Composition of Reaction Mixture

| Reagents | Amounts | |
|---|---|---|
| 10x PCR buffer | 2.5 | μl |
| 10% DMSO | 2.5 | μl |
| 10 mM dNTP mix | 0.5 | μl |
| 50 mM MgCl₂ | 0.75 | μl |
| Primer C (100 μM) | 0.125 | μl |
| aatB2 primer (100 μM) | 0.125 | μl |
| Colony culture medium (template) | 1.0 | μl |
| SDW | 17.38 | μl |
| Taq DNA polymerase recombinant (5 U/μl) | 0.125 | μl |
| Total | 25.0 | μl |

TABLE 8
PCR reaction program

| | | |
|---|---|---|
| 94° C. | 3 | minutes |

followed by 30 cycles under the following conditions

| | | |
|---|---|---|
| 94° C. | 45 | seconds, |
| 55° C. | 30 | seconds, |
| 72° C. | 3 | minutes, |

| | |
|---|---|
| 4° C. | To store |

The PCR product obtained by the colony PCR method was confirmed for the formation of the given entry clone by separating 5 μl from the reaction product onto 1.5% agarose gel.

Host Cells

The invention also relates to host cells comprising one or more of the nucleic acid molecules or vectors of the invention, particularly those nucleic acid molecules and vectors described in detail herein. Representative host cells that may be used according to this aspect of the invention include, but are not limited to, bacterial cells, yeast cells, plant cells and animal cells. Preferred bacterial host cells include Escherichia spp. cells (particularly E. coli cells and most particularly E. coli strains DH10B, Stbl2, DH5(, DB3, DB3.1 (preferably E. coli LIBRARY EFFICWENCY® DB3.1™ Competent Cells; Invitrogen Corporation, Carlsbad, Calif.), DB4 and DB5 (see U.S. application Ser. No. 09/518,188, filed Mar. 2, 2000, the disclosure of which is incorporated by reference herein in its entirety), Bacillus spp. cells (particularly B. subtilis and B. megaterium cells), Streptomyces spp. cells, Erwinia spp. cells, Klebsiella spp. cells, Serratia spp. cells (particularly S. marcessans cells), Pseudomonas spp. cells (particularly P. aeruginosa cells), and Salmonella spp. cells (particularly S. typhimurium and S. typhi cells). Preferred animal host cells include insect cells (most particularly Drosophila melanogaster cells, Spodoptera frugiperda Sf9 and Sf21 cells and Trichoplusa High-Five cells), nematode cells (particularly C. elegans cells), avian cells, amphibian cells (particularly Xenopus laevis cells), reptilian cells, and mammalian cells (most particularly NIH3T3, CHO, COS, VERO, BHK and human cells). Preferred yeast host cells include Saccharomyces cerevisiae cells and Pichia pastoris cells. These and other suitable host cells are available commercially, for example from Invitrogen Corporation (Carlsbad, Calif.), American Type Culture Collection (Manassas, Va.), and Agricultural Research Culture Collection (NRRL; Peoria, Ill.).

Methods for introducing the nucleic acid molecules and/or vectors of the invention into the host cells described herein, to produce host cells comprising one or more of the nucleic acid molecules and/or vectors of the invention, will be familiar to those of ordinary skill in the art. For instance, the nucleic acid molecules and/or vectors of the invention may be introduced into host cells using well known techniques of infection, transduction, electroporation, transfection, and transformation. The nucleic acid molecules and/or vectors of the invention may be introduced alone or in conjunction with other the nucleic acid molecules and/or vectors and/or proteins, peptides or RNAs. Alternatively, the nucleic acid molecules and/or vectors of the invention may be introduced into host cells as a precipitate, such as a calcium phosphate precipitate, or in a complex with a lipid. Electroporation also may be used to introduce the nucleic acid molecules and/or vectors of the invention into a host. Likewise, such molecules may be introduced into chemically competent cells such as E. coli. If the vector is a virus, it may be packaged in vitro or introduced into a packaging cell and the packaged virus may be transduced into cells. Hence, a wide variety of techniques suitable for introducing the nucleic acid molecules and/or vectors of the invention into cells in accordance with this aspect of the invention are well known and routine to those of skill in the art. Such techniques are reviewed at length, for example, in Sambrook, J., et al., Molecular Cloning, a Laboratory Manual, 2nd Ed., Cold Spring Harbor, N.Y.: Cold Spring Harbor Laboratory Press, pp. 16.30-16.55 (1989), Watson, J. D., et al., Recombinant DNA, 2nd Ed., New York: W. H. Freeman and Co., pp. 213-234 (1992), and Winnacker, E. -L., From Genes to Clones, New York: VCH Publishers (1987), which are illustrative of the many laboratory manuals that detail these techniques and which are incorporated by reference herein in their entireties for their relevant disclosures.

Polymerases

Polymerases for use in the invention include but are not limited to polymerases (DNA and RNA polymerases), and reverse transcriptases. DNA polymerases include, but are not limited to, Thermus thermophilus (Tth) DNA polymerase, Thermus aquaticus (Taq) DNA polymerase, Thermotoga neopolitana (Tne) DNA polymerase, Thermotoga maritima (Tma) DNA polymerase, Thermococcus litoralis (Tli or VENT™ ) DNA polymerase, Pyrococcus furiosus (Pfu) DNA polymerase, DEEPVENT™ DNA polymerase, Pyrococcus woosii (Pwo) DNA polymerase, Pyrococcus sp KOD2 (KOD) DNA polymerase, Bacillus sterothermophilus (Bst) DNA polymerase, Bacillus caldophilus (Bca) DNA polymerase, Sulfolobus acidocaldarius (Sac) DNA polymerase, Thermoplasma acidophilum (Tac) DNA polymerase, Thermus flavus (Tfl/Tub) DNA polymerase, Thermus ruber (Tru) DNA polymerase, Thermus brockianus (DYNAZYME™ ) DNA polymerase, Methanobacterium thermoautotrophicum (Mth) DNA polymerase, mycobacterium DNA polymerase (Mtb, Mlep), E. coli pol I DNA polymerase, T5 DNA polymerase, T7 DNA polymerase, and generally pol I type DNA polymerases and mutants, variants and derivatives thereof. RNA polymerases such as T3, T5, T7 and SP6 and mutants, variants and derivatives thereof may also be used in accordance with the invention.

The nucleic acid polymerases used in the present invention may be mesophilic or thermophilic, and are preferably thermophilic. Preferred mesophilic DNA polymerases include Pol I family of DNA polymerases (and their respective Kienow fragments) any of which may be isolated from organism such as E. coli, H. influenzae, D. radiodurans, H. pylori, C. aurantiacus, R. prowazekii, T. pallidum, Synechocystis sp., B. subtilis, L. lactis, S. pneumoniae, M. tuberculosis, M. leprae, M. smegmatis, Bacteriophage L5, phi-C31, T7, T3, T5, SP01, SP02, mitochondrial from S. cerevisiae MIP-1, and eukaryotic C. elegans, and D. melanogaster (Astatke, M. et al., 1998, J. Mol. Biol. 278, 147-165), pol III type DNA polymerase isolated from any sources, and mutants, derivatives or variants thereof, and the like. Preferred thermostable DNA polymerases that may be used in the methods and compositions of the invention include Taq, Tne, Tma, Pfu, KOD, Tfl, Tth, Stoffel fragment, VENT™ and DEEPVENT™ DNA polymerases, and mutants, variants and derivatives thereof (U.S. Pat. No. 5,436,149; U.S. Pat. No. 4,889,818; U.S. Pat. No. 4,965,188; U.S. Pat. No. 5,079,352; U.S. Pat. No. 5,614,365; U.S. Pat. No. 5,374,553; U.S. Pat. No. 5,270,179; U.S. Pat. No. 5,047,342; U.S. Pat. No. 5,512,462; WO 92/06188; WO 92/06200; WO 96/10640; WO 97/09451; Barnes, W. M., Gene 112:29-35 (1992); Lawyer, F. C., et al., PCR Meth. Appl. 2:275-287 (1993); Flaman, J. -M, et al., Nucl. Acids Res. 22(15):3259-3260 (1994)).

Kits

In another embodiment, the present invention may be assembled into kits, which may be used in the practice of the methods of the invention. Kits according to this aspect of the invention comprise a carrier means, such as a box, carton, tube or the like, having in close confinement therein one or more container means, such as vials, tubes, ampoules, bottles and the like, wherein a first container means contains one or more polypeptides (e.g., one or more recombination proteins, one or more topoisomerases, one or more restriction enzymes). The kits of the invention may also comprise (in the same or separate containers) one or more DNA polymerases, a suitable buffer, one or more nucleotides and/or one or more primers. The kits of the invention may also comprise one or more hosts or cells including those that are competent to take up nucleic acids (e.g., DNA molecules including vectors). Preferred hosts may include chemically competent or electrocompetent bacteria such as E. coli (including DH5, DH5α, DH10B, HB101, Top 10, and other K-12 strains as well as E. coli B and E. coli W strains).

It is to be noted that although the present invention will be described in more detail by way of examples, the present invention is not limited to the examples. As the enzymes and reagents used in the examples, there were illustrated those of an analysis level and a biochemical level as commercially available from Invitrogen Company or Wako Jyunyak K. K., unless otherwise stated.

### EXAMPLE 1

This example is directed to an embodiment of the formation of a GATEWAY™ entry clone by using a mixture of primers B and B′ having each a sequence of bases, ATT and ATA, respectively, hybridizable with the stop codon of a template cDNA as a primer having an adapter b at the 3′-terminus in the first step of the PCR method.

The GATEWAY™ entry clone was formed by subjecting DNA segments containing cDNAs having ORF 624, 1473, 915, 1389, 498, 1410, 378 and 672, respectively, found in the human full length cDNA project, to amplification of cDNAs by the two-step adapter PCR. The first step of the PCR method was carried out by means of the PCR method basically by using the primer A and a mixture of the primers B and B′ having the sequences of bases, 3′-ATT-5′ and 3′-ATA-5′, respectively, hybridizable with the stop codon of the template cDNA having the adapter b as described in SEQ ID NO. 2 and using the composition of the reaction mixture as indicated in Table 2 above under the reaction program as indicated in Table 3 above. In the first step and the second step of the PCR method, a review was made regarding two different kinds of DNA polymerases, i.e., PLATINUM Taq DNA polymerase Hligh Fidelity (hereinafter referred to as “Taq HiFi”) and PLATINUM Pfx DNA polymerase (hereinafter referred to as “Pfx”) as indicated in Tables 2 and 4, respectively. Further, a review on the case of an addition of an enhancer solution (Invitrogen Corporation, Carlsbad, Calif. catalog # 11495017) and on the case of no addition thereof was made for the case where the Taq HiFi was used.

The first step of the PCR method was carried out by using a 96-well plate and preparing a reaction mixture (for 124 reactions) having the composition as indicated in Table 9 below and pouring 20 μl of the reaction mixture into each tube with a 12-channel pipette. Thereafter, the primer A (10 μM) in the amount of 0.5 μl, a mixture of the primers B and B′ (10 μM) in the amount of 0.5 μl, and the template DNA (50 ng˜) in the amount of 5.0 μl were added to each tube, and the reaction was conducted under the reaction program as indicated in Table 3 above.

TABLE 9
Composition of Reaction Mixture (for 96-well plate)
DNA polymerase

| | Taq HiFi (5 units/μl) | | | | | |
|---|---|---|---|---|---|---|
| Enhancer Solution | | | No Addition | | Pfx (2.5 units/μl) | |
| Reagents | Addition | | Amounts | | Addition | |
| 10x PCR buffer | 310 | μl | 310 | μl | 310 | μl |
| Enhancer solution | 310 | μl | — | | 310 | μl |
| 10 mM dNTP mix | 62 | μl | 62 | μl | 15.5 | μl |
| 50 mM MgSO₄ | 124 | μl | 124 | μl | 93 | μl |
| SDW | 1537.6 | μl | 1835.2 | μl | 1540.7 | μl |
| DNA polymerase | 12.4 | μl | 24.8 | μl | 24.8 | μl |
| Total | 2356 | μl | 2356 | μl | 2293 | μl |

The second step of the PCR method was carried out by means of the PCR basically by using the primer C having the sequence of bases as described in SEQ ID NO. 6 and the primer D having the sequence of bases as described in SEQ ID NO. 7 or the primer D′ having the sequence of bases as described in SEQ ID NO. 8 and using the reaction mixture having the composition as indicated in Table 4 above under the reaction program as indicated in Table 5 above.

The second step of the PCR method was carried out in substantially the same manner as in the first step thereof by using a 96-well plate and a reaction mixture (for 124 reactions) having the composition as indicated in Table 10 and pouring 20 μl of the reaction mixture into each tube with a 12-channel pipette. Thereafter, 5.0 μl of the PCR product obtained in the first step was added to each tube, and the reaction was conducted under the reaction program as indicated in Table 5 above.

TABLE 10
Composition of Reaction Mixture (for 96-well plate)
DNA polymerase

| | Taq HiFi (5 units/μl) | | | | | |
|---|---|---|---|---|---|---|
| Enhancer Solution | | | No Addition | | Pfx (2.5 units/μl) | |
| Reagents | Addition | | Amounts | | Addition | |
| 10x PCR buffer | 310 | μl | 310 | μl | 310 | μl |
| Enhancer solution | 310 | μl | — | | 310 | μl |
| 10 mM dNTP mix | 62 | μl | 62 | μl | 15.5 | μl |
| 50 mM MgSO₄ | 124 | μl | 124 | μl | 93 | μl |
| Primer C (100 μM) | 24.8 | μl | 24.8 | μl | 37.2 | μl |
| Primer D or D′ (100 μM) | 24.8 | μl | 24.8 | μl | 37.2 | μl |
| SDW | 1612 | μl | 1922 | μl | 1552.3 | μl |
| DNA polymerase | 12.4 | μl | 24.8 | μl | 24.8 | μl |
| Total | 2480 | μl | 2480 | μl | 2480 | μl |

As a result of the reaction under the two-step adapter PCR method, the PCR products of the native-type cDNA and the C-terminal fused-type cDNA, each having the sequence attB1 at the 5′-terminus and the sequence attB2 at the 3′-terminus. The integration into the attP plasmid was carried out under the BP reaction for the formation of an entry clone. The BP reaction was conducted basically by using the reaction mixture having the composition as indicated in Table 6 above and by using a 96-well plate and preparing the reaction mixture (for 124 reactions) having the composition as indicated in Table 11 above in substantially the same manner as in the PCR.

TABLE 11
Composition of BP Reaction Mixture (for 96-well plate)

| Reagents | Amounts | |
|---|---|---|
| 5x PCR reaction buffer | 248 | μl |
| pDONR201 (150 ng/μl) | 124 | μl |
| BP CLONASE enzyme mixed solution | 248 | μl |
| SDW | 24.8 | μl |
| Total | 644.8 | μl |

The operations of the BP reaction were conducted by mixing the reaction mixture as indicated in Table 11 above on ice, pouring 5 μl of the reaction mixture into each tube, adding thereto the second PCR product in the amount of 5 μl, incubating the resulting reaction mixture overnight at 25° C., followed by adding 1 μl of 10×proteinase K (2 μg/μl) to the incubated reaction mixture and terminating the reaction by further incubating the reaction mixture at 37° C. for 10 minutes.

The GATEWAY™ entry clone was formed by transforming a competent cell with the product obtained by the BP reaction under the operations as will be described hereinafter. The BP reaction product was added in the amount of 5 μl to 50 μl of a solution of the competent cell (DH5α) on ice, and the cell was then incubated intact for 30 minutes, subjected to heat shock at 42° C. and immediately thereafter transferred onto ice and allowed to stand for 2 minutes. Then, 250 μl of a SOC culture medium was added thereto and subjected to shaking culture for 1.5 hours at 37° C. The culture medium in the amount of 100 μl to 150 μl was then inoculated on a selection culture medium plate (an LB culture medium plate containing kanamycin at the rate of 50 μl/ml of) and incubated at 37° C.

The number of the colonies obtained for each clone was confirmed under the following experiments. The results of confirmation will be shown in Table 12 below.

TABLE 12

| | DNA Polymerase in the first and second PCR steps | | | | | |
|---|---|---|---|---|---|---|
| | with Addition or No Addition of Enhancer Solution | | | | | |
| | Taq HiFi | | | | | |
| | | | No Addition of | | Pfx | |
| | Addition of Enhancer | | Enhancer | | Addition of Enhancer | |
| | | C-terminal | | C-terminal | | C-terminal |
| Clone | Native- | Fused- | Native- | Fused- | Native- | Fused- |
| No. | Type | Type | Type | Type | Type | Type |
| Clone 1 | 6124 | 3515 | 6000 | 6200 | 5500 | 6200 |
| (ORF 624) | | | | | | |
| Clone 2 | 3629 | 3005 | 1400 | 3200 | 5500 | 6500 |
| (ORF 1473) | | | | | | |
| Clone 3 | 3175 | 3912 | 2600 | 5200 | 5400 | 6600 |
| (ORF 915) | | | | | | |
| Clone 4 | 794 | 1871 | 1900 | 2900 | 6000 | 5700 |
| (ORF 1389) | | | | | | |
| Clone 5 | 1985 | 2778 | 5400 | 3100 | 6900 | 7800 |
| (ORF 498) | | | | | | |
| Clone 6 | 1134 | 1304 | 1 | 4 | 3200 | 2000 |
| (ORF 1410) | | | | | | |
| Clone 7 | 4309 | 6691 | 5800 | 6700 | 5000 | 4900 |
| (ORF 378) | | | | | | |
| Clone 8 | 3742 | 3629 | 6700 | 3600 | 7700 | 6000 |
| (ORF 672) | | | | | | |

It was found from the results as shown in Table 12 above that a sufficient number of colonies can be obtained for each clone under the reaction conditions of the two-step PCR. Four colonies were selected optionally from the grown colonies of each clone, and subjected to shaking culture in 0.2 ml of a TB culture medium containing kanamycin at the rate of 50 μg/ml. Thereafter, 50 μl of the resulting culture medium was added to a 80% glycerol solution, stirred well and sealed, thereafter storing the mixture at −80° C.

A confirmation was made as to the formation of the objective entry clone for the selected colonies of each clone. The colonies obtained from each clone and stored were then subjected to the amplification of cDNA by means of the colony PCR. The colony PCR was carried out basically by using the reaction mixture having the composition as indicated in Table 7 above under the reaction program as indicated in Table 8 above.

The colony PCR was carried out by using a 284-well plate in substantially the same manner as the first and second steps of the PCR. The colony PCR was carried out under the reaction program as indicated in Table 8 above by preparing the reaction mixture (for 416 reactions) having the composition as indicated in Table 13 and pouring 15 μl of the reaction mixture into each tube with a 12-channel pipette, followed by adding 0.5 μl of the colony culture medium to each tube.

TABLE 13
Composition of BP Reaction Mixture (for 284-well plate
and 416 reactions)

| Reagents | Amounts | |
|---|---|---|
| 10x PCR buffer | 624 | μl |
| Enhancer solution | 624 | μl |
| 10 mM dNTP mix | 124.8 | μl |
| 50 mM MgCl₂ | 249.6 | μl |
| Primer C (100 μM) | 37.44 | μl |
| attB2 Primer (100 μM) | 37.44 | μl |
| SDW | 4517.76 | μl |
| Taq DNA polymerase recombinant | 52 | μl |
| (5 units/μl) | | |
| Total | 6267 | μl |

A confirmation was made for the sequence of bases of the stop codons for the four colonies of each clone confirmed by the colony PCR, and the results of confirmation will be shown in Table 14 below. The transformation of the stop codon by the two-step adapter PCR was indicated as a number of the colonies having the objective sequence out of the four colonies optionally selected.

TABLE 14

| | DNA Polymerase in the first and second PCR steps | | | | | |
|---|---|---|---|---|---|---|
| Clone | with Addition or No Addition of Enhancer Solution | | | | | |
| No. | Taq HiFi | | | | Pfx | |
| (Original | Addition of Enhancer | | No Addition of Enhancer | | Addition of Enhancer | |
| stop | Native- | C-terminal | Native- | C-terminal | Native- | C-terminal |
| codon) | Type | Fused-Type | Type | Fused-Type | Type | Fused-Type |
| Clone 1 | 4/4 | 4/4 | 4/4 | 4/4 | 4/4 | 4/4 |
| (ORF 624) | | | | | | |
| (TGA) | | | | | | |
| Clone 2 | 4/4 | 4/4 | 4/4 | 4/4 | 2/4 | 2/4 |
| (ORF 1473) | | | | | | |
| (TAA) | | | | | | |
| Clone 3 | 4/4 | 4/4 | 3/4 | 4/4 | 3/4 | 4/4 |
| (ORF 915) | | | | | | |
| (TAA) | | | | | | |
| Clone 4 | 4/4 | 4/4 | 4/4 | 4/4 | 2/4 | 4/4 |
| (ORF 1389) | | | | | | |
| (TGA) | | | | | | |
| Clone 5 | 4/4 | 4/4 | 4/4 | 4/4 | 4/4 | 4/4 |
| (ORF 498) | | | | | | |
| (TAA) | | | | | | |
| Clone 6 | 4/4 | 4/4 | — | — | 4/4 | 2/4 |
| (ORF 1410) | | | | | | |
| (TGA) | | | | | | |
| Clone 7 | 4/4 | 4/4 | 4/4 | 3/4 | 4/4 | 4/4 |
| (ORF 378) | | | | | | |
| (TAA) | | | | | | |
| Clone 8 | 4/4 | 4/4 | 4/4 | 4/4 | 3/4 | 2/4 |
| (ORF 672) | | | | | | |
| (TGA) | | | | | | |

It was found from the results as shown in Table 14 above that the clones of the native-type cDNA and the C-terminal fused-type cDNA were obtained simultaneously and highly selectively in the formation of the entry Clone by the two-step adapter PCR according to the present invention. In particular, when the enhancer solution was added to Taq HiFi, it was found that both of the native-type cDNA and the C-termninal fused-type cDNA were cloned with a 100% selectivity.

### EXAMPLE 2

This example relates to an embodiment of the formation of a GATEWAY™ entry clone by using a mixture of primers B and B′ having each a sequence of bases, 3′-ACT-5′ and 3-ACA-5′, respectively, hybridizable with the stop codon of a template cDNA as a primer and having an adapter b at the 5′-terminus of the primer in the first step of the PCR method.

The GATEWAY™ entry clone was formed by subjecting DNA segments containing cDNAs having ORF 624, 1473, 915, 1389, 498, 1410, 378 and 672, respectively, selected in the human full length cDNA project, to the amplification of cDNAs by the two-step adapter PCR in substantially the same manner as in Example 1 above. The first step of the PCR method was carried out by means of the PCR basically by using the primer A and a mixture of the primers B and B′ having the sequences of bases, 3′-ACT-5′ and 3′-ACA-5′, respectively, hybridizable with the stop codon of the template cDNA and having the adapter b as described in SEQ ID NO. 2 and using the reaction mixture having the composition as indicated in Table 2 above under the reaction program as indicated in Table 3 above. A review was made regarding two different kinds of DNA polymerases, i.e., Taq HiFi and Pfx as indicated in Tables 2 and 4, respectively, in the first step and the second step of the PCR method.

The first step of the PCR method was carried out in substantially the same manner as in Example 1 by using a 96-well plate and preparing a reaction mixture (for 124 reactions) having the composition as indicated in Table 9 above under the reaction program as indicated in Table 3 above. In the case where Taq HiFi was used, however, the reaction without addition of the enhancer solution was not carried out in this Example.

The second step of the PCR method was carried out in substantially the same manner as in Example 1 by using a 96-well plate and preparing the reaction mixture (for 124 reactions) having the composition as indicated in Table 10 under the reaction program as indicated in Table 5 above.

The second step of the PCR method was carried out by means of the PCR basically by using the reaction mixture having the composition as indicated in Table 4 above under the reaction program as indicated in Table 5 above and using the primer C having the sequence of bases as described in SEQ ID NO. 4 and the primer D having the sequence of bases as described in SEQ ID NO. 9 or the primer D′ having the sequence of bases as described in SEQ ID NO. 10.

The BP reaction for the formation of the entry clone was carried out in substantially the same manner as in Example 1 for the PCR products of the native-type cDNA and the C-terminal fused-type cDNA obtained in the two-step adapter PCR method.

The GATEWAY™ entry clone was formed by transforming the competent cells with the product obtained by the BP reaction in substantially the same manner as in Example 1. Table 15 below shows the results of confirmation of the number of the colonies obtained for each clone under the experiments as conducted above.

TABLE 15

| | DNA Polymerase in the first and second PCR steps | | | | | |
|---|---|---|---|---|---|---|
| | with Addition or No Addition of Enhancer Solution | | | | | |
| | Taq HiFi | | | | Pfx | |
| | Addition of Enhancer | | No Addition of Enhancer | | Addition of Enhancer | |
| Clone | Native- | C-terminal | Native- | C-terminal | | C-terminal |
| No. | Type | Fused-Type | Type | Fused-Type | Native-Type | Fused-Type |
| Clone 1 | 794 | 907 | — | — | 1000 | 1700 |
| (ORF 624) | | | | | | |
| Clone 2 | 193 | 624 | — | — | 500 | 1300 |
| (ORF 1473) | | | | | | |
| Clone 3 | 340 | 794 | — | — | 1400 | 1200 |
| (ORF 915) | | | | | | |
| Clone 4 | 119 | 510 | — | — | 1400 | 1500 |
| (ORF 1389) | | | | | | |
| Clone 5 | 851 | 1304 | — | — | 2500 | 2300 |
| (ORF 498) | | | | | | |
| Clone 6 | 94 | 143 | — | — | 1800 | 1600 |
| (ORF 1410) | | | | | | |
| Clone 7 | 1134 | 1418 | — | — | 1200 | 2200 |
| (ORF 378) | | | | | | |
| Clone 8 | 1474 | 1191 | — | — | 1100 | 1200 |
| (ORF 672) | | | | | | |

It was found from the results as shown in Table 15 above that a sufficient number of colonies can be obtained for each clone under the reaction conditions of the two-step PCR. Four colonies were selected optionally from the grown colonies of each clone in substantially the same manner as in Example 1, and stored at −80 ° C. after they were subjected to the necessary treatment.

Thereafter, the confirmation was carried out in substantially the same manner as in Example 1 by the colony PCR as to whether the objective entry clone was formed for the four colonies of each clone confirmed by the colony PCR.

Table 16 below shows the results of confirmation for the sequence of bases of the stop codons in respect of the four colonies of each clone confirmed by the colony PCR. The transformation of the stop codons by the two-step adapter PCR was indicated as a number of the colonies having the objective sequence with respect to the four colonies optionally selected.

TABLE 16

| | DNA Polymerase in the first and second PCR steps | | | | | |
|---|---|---|---|---|---|---|
| Clone | with Addition or No Addition of Enhancer Solution | | | | | |
| No. | Taq HiFi | | | | Pfx | |
| (Original | Addition of Enhancer | | No Addition of Enhancer | | Addition of Enhancer | |
| termination | Native- | C-terminal | Native- | C-terminal | Native- | C-terminal |
| codon) | Type | Fused-Type | Type | Fused-Type | Type | Fused-Type |
| Clone 1 | 4/4 | 4/4 | — | — | 4/4 | 4/4 |
| (ORF 624) | | | | | | |
| (TGA) | | | | | | |
| Clone 2 | 4/4 | 4/4 | — | — | 3/4 | 4/4 |
| (ORF 1473) | | | | | | |
| (TAA) | | | | | | |
| Clone 3 | 4/4 | 3/4 | — | — | 4/4 | 3/4 |
| (ORF 915) | | | | | | |
| (TAA) | | | | | | |
| Clone 4 | 3/4 | 4/4 | — | — | 4/4 | 3/4 |
| (ORF 1389) | | | | | | |
| (TGA) | | | | | | |
| Clone 5 | 4/4 | 4/4 | — | — | 4/4 | 3/4 |
| (ORF 498) | | | | | | |
| (TAA) | | | | | | |
| Clone 6 | 4/4 | 4/4 | — | — | 4/4 | 4/4 |
| (ORF 1410) | | | | | | |
| (TGA) | | | | | | |
| Clone 7 | 4/4 | 4/4 | — | — | 3/4 | 3/4 |
| (ORF 378) | | | | | | |
| (TAA) | | | | | | |
| Clone 8 | 4/4 | 4/4 | — | — | 4/4 | 4/4 |
| (ORF 672) | | | | | | |
| (TGA) | | | | | | |

It was found from the results as shown in Table 16 above that the clones of the native-type cDNA and the C-terminal fused-type cDNA were obtained simultaneously and highly selectively in the formation of the entry clone by the two-step adapter PCR according to the present invention.

### EXAMPLE 3

This example relates to an embodiment of the formation of a GATEWAY™ entry clone by using a mixture of primers B and B′ having each a sequence of bases, 3′-ACT-5′ and 3′-CCT-5′, respectively, hybridizable with the stop codon of a template cDNA as a primer and having an adapter b at the 5′-terminus of the primer in the first step of the PCR method.

The GATEWAY™ entry clone was formed by subjecting DNA segments containing cDNAs having ORF 624, 1473, 915, 1389, 498, 1410, 378 and 672, respectively, selected in the human full length cDNA project, to the amplification of cDNAs by the two-step adapter PCR in substantially the same manner as in Example 1 above. The first step of the PCR method was carried out by means of the PCR basically by using the primer A and a mixture of the primers B and B′ having the sequences of bases, 3′-ACT-5′ and 3′-CCT-5′, respectively, hybridizable with the stop codon of the template cDNA and having the adapter b as described in SEQ ID NO. 2 and using the reaction mixture having the composition as indicated in Table 2 above under the reaction program as indicated in Table 3 above. A review was made regarding two different kinds of DNA polymerases, i.e., Taq HiFi and Pfx as indicated in Tables 2 and 4, respectively, in the first step and the second step of the PCR method. Further, in the case where Taq HiFi was used, a review was made regarding the case where the enhancer solution was added and the case where no enhancer solution was added.

The first step of the PCR method was carried out in substantially the same manner as in Example I by using a 96-well plate and preparing a reaction mixture (for 124 reactions) having the composition as indicated in Table 9 above under the reaction program as indicated in Table 3 above.

The second step of the PCR method was carried out by means of the PCR in substantially the same manner as in Example 1 above basically by using the reaction mixture having the composition as indicated in Table 4 above under the reaction program as indicated in Table 5 above and using the primer C having the sequence as described in SEQ ID NO. 4 and the primer D having the sequence as described in SEQ ID NO. 9 or the primer D′ having the sequence as described in SEQ ID NO. 11.

The BP reaction for the formation of the entry clone was carried out in substantially the same manner as in Example 1 for the PCR products of the native-type cDNA and the C-terminal fused-type cDNA obtained in the twostep PCR method.

The GATEWAY™ entry clone was formed by transforming the competent cells with the products obtained by the BP reaction in substantially the same manner as in Example 1.

Table 17 below shows the results of confirmation of the number of colonies obtained for each clone.

TABLE 17

| | DNA Polymerase in the first and second PCR steps | | | | | |
|---|---|---|---|---|---|---|
| | with Addition or No Addition of Enhancer Solution | | | | | |
| | Taq HiFi | | | | Pfx | |
| | Addition of Enhancer | | No Addition of Enhancer | | Addition of Enhancer | |
| Clone | Native- | C-terminal | Native- | C-terminal | Native- | C-terminal |
| No. | Type | Fused-Type | Type | Fused-Type | Type | Fused-Type |
| Clone 1 | 5046 | 4990 | 9200 | 7800 | 6600 | 5100 |
| (ORF 624) | | | | | | |
| Clone 2 | 3005 | 2722 | 3600 | 2500 | 7400 | 5400 |
| (ORF 1473) | | | | | | |
| Clone 3 | 4763 | 3572 | 5200 | 6700 | 7400 | 8100 |
| (ORF 915) | | | | | | |
| Clone 4 | 3062 | 1985 | 4200 | 3400 | 7600 | 5700 |
| (ORF 1389) | | | | | | |
| Clone 5 | 2665 | 3119 | 8100 | 5800 | 6900 | 7800 |
| (ORF 498) | | | | | | |
| Clone 6 | 1361 | 737 | 6 | 1 | 1200 | 3300 |
| (ORF 1410) | | | | | | |
| Clone 7 | 6237 | 2835 | 8800 | 7900 | 8000 | 6800 |
| (ORF 378) | | | | | | |
| Clone 8 | 5330 | 4423 | 6200 | 8200 | 7200 | 7200 |
| (ORF 672) | | | | | | |

It was found from the results as shown in Table 17 above that a sufficient number of the colonies can be obtained for each clone under the reaction conditions of the two-step PCR. Four colonies were selected optionally from the grown colonies of each clone and stored at −80° C. after they were subjected to the necessary treatments.

The confirmation was made by the colony PCR as to whether the objective entry clone was formed for the four colonies of each clone selected. The colony PCR was carried out in substantially the same manner as in Example 1.

Table 18 below shows the results of confirmation for the sequence of bases of the stop codons in respect of the four colonies of each clone confirmed by the colony PCR. The transformation of the stop codons by the two-step adapter PCR was indicated as a number of the colonies having the objective sequence with respect to the four colonies optionally selected.

TABLE 18

| | DNA Polymerase in the first and second PCR steps | | | | | |
|---|---|---|---|---|---|---|
| Clone | with Addition or No Addition of Enhancer Solution | | | | | |
| No. | Taq HiFi | | | | Pfx | |
| (Original | Addition of Enhancer | | No Addition of Enhancer | | Addition of Enhancer | |
| termination | Native- | C-terminal | Native- | C-terminal | Native- | C-terminal |
| codon) | Type | Fused-Type | Type | Fused-Type | Type | Fused-Type |
| Clone 1 | 4/4 | 4/4 | 4/4 | 4/4 | 1/4 | 1/4 |
| (ORF 624) | | | | | | |
| (TGA) | | | | | | |
| Clone 2 | 4/4 | 4/4 | 3/4 | 4/4 | 0/4 | 4/4 |
| (ORF 1473) | | | | | | |
| (TAA) | | | | | | |
| Clone 3 | 4/4 | 3/4 | 3/4 | 4/4 | 0/4 | 4/4 |
| (ORF 915) | | | | | | |
| (TAA) | | | | | | |
| Clone 4 | 4/4 | 4/4 | 3/4 | 4/4 | 1/4 | 3/4 |
| (ORF 1389) | | | | | | |
| (TGA) | | | | | | |
| Clone 5 | 4/4 | 4/4 | 3/4 | 3/4 | 1/4 | 3/4 |
| (ORF 498) | | | | | | |
| (TAA) | | | | | | |
| Clone 6 | 4/4 | 4/4 | — | — | 2/4 | 4/4 |
| (ORF 1410) | | | | | | |
| (TGA) | | | | | | |
| Clone 7 | 4/4 | 4/4 | 4/4 | 4/4 | 3/4 | 3/4 |
| (ORF 378) | | | | | | |
| (TAA) | | | | | | |
| Clone 8 | 4/4 | 4/4 | 4/4 | 3/4 | 1/4 | 2/4 |
| (ORF 672) | | | | | | |
| (TGA) | | | | | | |

It was found from the results as shown in Table 18 above that the clones of the native-type cDNA and the C-terminal fused-type cDNA were obtained simultaneously and highly selectively in the formation of the entry clone by the two-step adapter PCR according to the present invention. In particular, it was found that both of the native-type cDNA and the C-terminal fused-type cDNA were cloned with nearly 100% selectivity in the case where the enhancer solution was added when Taq HiFi was used as a DNA polymerase.

The method for the preparation of the GATEWAY™ entry clone according to the present invention enables the rapid and easy formation of multiple kinds of expressed clones. Further, this method can contribute to the ready analysis of functions of cDNA and PCR DNA, the ready expression of proteins and so on as well as the development of pharmaceutical compounds.

### EXAMPLE 4

The methods of the invention may be practiced using a single-tube reaction. Thus, primers A, B, B′, C, D, and D′ may be added to a single reaction tube and a single PCR reaction used to create the nucleic acids of the invention.

In methods of this type, the portion of primers B and B′ that are not specific to the sequence of the templates (i.e., adapter sequences b and b′) may have a different sequence from each other. Making use of the redundancy of the genetic code, b and b′ may still encode the same amino acids. The sequences of primers D and D′ may be selected such that they anneal to the appropriate PCR product (e.g., by making use of the differences between b and b′). Primers D and D′ may also comprise other sequences (e.g., recombination sites, topoisomerase recognition sites, etc.). In a specific embodiment, primer D may comprise a first attB sequence while primer D′ may comprise a different attB sequence.

The sequences of primers B and B′ may be selected such that they anneal to the template at a temperature that is higher than the temperature at which primers D and D′ anneal to the extension products of the B and B′ primers. A PCR reaction may be run at a first temperature high enough to prevent the primers D and D′ from annealing. After the initial phase of the reaction, which may comprise from 1 to about 25, from 1 to about 20, from 1 to about 15, from 1 to about 10, from 1 to about 9, from 1 to about 8, from 1 to about 7, from 1 to about 6, from 1 to about 5, from 1 to about 4, from 1 to about 3 or from 1 to about 2 cycles, the annealing temperature of the reaction may be reduced to a point at which primers D and D′ can anneal to the extension products of primers B and B′. After a suitable number of cycles, (e.g., from about 5 to about 50, from about 5 to about 40, from about 5 to about 30, from about 5, to about 25, from about 5, to about 20, from about 5 to about 15, from about 5 to about 10, from about 5 to about 9, from about 5 to about 8, from about 5 to about 7, or from about 5 to about 6), the PCR product can be used in a cloning reaction (e.g., a recombinational cloning, a topisomerase-mediated cloning and/or a ligase-mediated cloning) as described above.

In some embodiment, the PCR products containing different recombination sites may be contacted with vectors that comprise the appropriate recombination sites to react with one or another of the PCR products. For example, one PCR product may be comprise an attB1 sequence at one end and an attB2 sequence at the other end. Another PCR product may have an attB1 site at one end and an attB5 site at the other end. Vectors may be provided to react with one of the PCR products and preferably not the other. For example one vector may comprise an attPI site and an attP2 site while the other vector may comprise an attPI site and an attP5 site. Optionally the vectors may provide resistance to different toxic compounds, for example, one vector may provide resistance to a first antibiotic (e.g., ampicillin) while the other provides resistance against a second antibiotic (e.g., chloramphenicol).

In some embodiments, two nested PCR reactions may be set up to prepare the nucleic acid molecules of the invention. Both reactions may contain a pair of primers for each end of the target sequence. For example, with reference to FIG. 4, both PCR reactions may contain primer A, which may comprise sequences that hybridize to the sequence of interest and an adapter sequence a, and primer C, which may comprise sequences that hybridize to the adapter sequence a and also may comprise adapter sequence c and may also comprise sequences that hybridize to the sequence of interest. Both PCR reactions may also contain a mixture of primers B and B′, both of which may comprise sequences that hybridize to the sequence of interest and an adapter sequence b. As discussed above, the sequences of the adapters may be the same or different. A first PCR reaction (Tube 1 in FIG. 4) may contain primer D, which may comprise sequences that hybridize to adapter sequence b and may also comprise adapter sequence d. A second PCR reaction (Tube 2 in FIG. 4) may contain primer D′, which may comprise sequences that hybridize to adapter sequence b and may also comprise adapter sequence d. In some embodiments, primers D and D′ may anneal to the PCR product produced by extension of primers B and B with the 3′-most nucleotide of primer D or D′ aligned with one of the nucleotides of the stop codon.

The ratio of the concentration of primers in the primer pairs for each of the sequence of interest (i.e., the ratio of [primer A]:[primer C], [primer B+B′]:[primer D], and [primer B+B′]:[primer D′]) may varied to optimize the production of the desired PCR product. The ration may range from about 25:1 to about 1:25, from about 20:1 to about 1:20, from about 15:1 to about 1:15, from about 10:1 to about 1:10, from about 5:1 to about 1:5 or may be about 1:1. In some embodiments, the ratio of [primer B+B′]:[primer D or D′] may be about 1:10. The total concentration of primers in a primer pair may be from about 0.001 μM to about 100 μM, from about 0.01 μm to about 50 μM, from about 0.1 μM to about 25 μM, from about 0.1 μM to about 10 μM, from about 0.1 μM to about 5.0 μM, from about 0.1 μM to about 1.0 μM, or from about 0.1 μM to about 0.5 μM.

In some embodiments, primers B and B′ may be made by degenerate synthesis (i.e. the synthesis reaction of the nucleotides corresponding to the stop codon may contain multiple nucleotides). For example, in some embodiments, primers B and B′ may be synthesized simultaneously by including both the nucleotide corresponding to the stop codon and a nucleotide corresponding to the mutation to be introduced in the same reaction.

In some embodiments, the sequences of primers B and B′ may be selected so as to change all stop codons into TAA stop codons and to change all mutations into TAT codons, which encode tyrosine. This can be accomplished by making the portion of the primer that anneals to the stop codon contain mis-matched nucleotides at the last two positions of the stop codon. For example, primer B may contain 3′-TT-5′ at the position corresponding to the last two positions of the stop codon while primer B′ may contain 3′-TA-5′ at this position.

Having now fully described the present invention in some detail by way of illustration and example for purposes of clarity of understanding, it will be obvious to one of ordinary skill in the art that the same can be -performed by modifying or changing the invention within a wide and equivalent range of conditions, formulations and other parameters without affecting the scope of the invention or any specific embodiment thereof, and that such modifications or changes are intended to be encompassed within the scope of the appended claims.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

What is claimed is:

1. A method of preparing a plurality of nucleic acid molecules, comprising:
contacting a template nucleic acid molecule with at least a first, second and third primer and a polypeptide having DNA polymerase activity to form a mixture;
incubating the mixture under conditions sufficient to extend the primers, wherein both the second and the third primers comprise a sequence that anneals to a sequence on the template and, wherein the sequence of at least one of the second and third primers comprises at least one base that does not base pair with the sequence of the template.

2. A method of amplifying a double-stranded DNA molecule comprising:
(a) providing at least a first, second and third primer, wherein the first primer is at least partially complementary to a sequence of the second strand of the DNA molecule and the second and third primers are at least partially complementary to a sequence of the first strand of the DNA molecule;
(b) hybridizing the first primer to the second strand and the second and third primers to the first strand in the presence of a polypeptide having DNA polymerase activity, under conditions such that a third DNA strand complementary to the second strand and a fourth and a fifth DNA strand complementary to the first strand are synthesized, wherein at least one of the second and third primers contains nucleotide that does not base pair to the sequence on the first strand of the DNA molecule to which it hybridizes.

3. A method according to claim 2, comprising:
(c) denaturing the product of (b); and
(d) repeating (a) to (c) one or more times to produce third strands comprising a sequence complementary to either the second or the third primer and to produce fourth and fifth strands comprising a sequence complementary to the first primer.

4. A method according to claim 3, wherein (a) to (c) are repeated from 1 to 25 times.

5. A method according to claim 3, wherein (a) to (c) are repeated from 1 to 15 times.

6. A method according to claim 3, wherein (a) to (c) are repeated from 3 to 10 times.

7. A method according to claim 3, wherein (a) to (c) are repeated from 3 to 8 times.

8. A method according to claim 3, wherein (a) to (c) are repeated 4 to 6 times.

9. A method according to claim 3, wherein the first primer comprises a first adapter sequence, the second primer comprises a second adapter sequence and/or the third primer comprises a third adapter sequence.

10. A method according to claim 9, wherein the adapter sequence of the second and the third primer are the same.

11. A method according to claim 9, wherein the adapter sequence of the second and the third primer are different.

12. A method according to claim 3, comprising:
(e) contacting the product of (d) with at least a fourth, fifth and sixth primer, wherein the fourth primer is at least partially complementary to the fourth and fifth strands and the fifth and sixth primers are at least partially complementary to the third strands; and
(f) hybridizing the fourth primer to the fourth and fifth strands and the fifth and sixth primers to the third strands in the presence of a polypeptide having DNA polymerase activity, under conditions such that a sixth DNA strand complementary to the fourth strand, a seventh DNA strand complementary to the fifth strand, an eighth DNA strand complementary to the thirds strands, and a ninth DNA strand complementary to the third strands are synthesized.

13. A method according to claim 12, comprising
(g) denaturing the product of (f); and
(h) repeating (e) to (g) one or more times to produce sixth strands comprising a sequence complementary to the fifth primer, seventh strands comprising a sequence complementary to the sixth primer, eighth strands comprising a sequence complementary to the fourth primer and ninth strands comprising a sequence complementary to the fourth primer.

14. A method according to claim 13, wherein at least one of the fourth, fifth or sixth primer comprises an adapter sequence.

15. A method according to claim 13, wherein the fourth primer comprises a fourth adapter sequence, the fifth primer comprises a fifth adapter sequence and the sixth primer comprises a sixth primer sequence.

16. A method according to claim 13, wherein the adapter sequences of the fifth and sixth primers are the same.

17. A method according to claim 13, wherein the adapter sequences of the fifth and sixth primers are different.

18. A method according to claim 15, wherein each adapter sequences comprises a sequence independently selected from the group consisting of restriction enzyme recognition sites, topoisomerase recognition sites, recombination sites, and transposition sites.

19. A method according to claim 18, wherein the recombination sites are selected from a group consisting of lox sites and att sites.

20. A method according to claim 18, wherein the att sites are selected from a group consisting of attB1, attP1, attL1, attR1, attB2, attP2, attL2, attR2, attB3, attP3, attL3, attR3, attB5, attP5, attL5, and attR5.

21. A method according to claim 13, comprising
(i) contacting the product of (h) with one or more polypeptides and one or more vectors under conditions sufficient to insert all or a portion of the product of (h) into the vector.

22. A method according to claim 21, comprising
(j) transforminng a competent cell with the product of (i); and
(k) selecting for cells comprising the product of (i).

23. A method according to claim 21, wherein one or more vectors comprise at least one recombination site and at least one polypeptide is a recombination protein.

24. A method according to claim 21, wherein the product of (h) is contacted with one or more polypeptides and at least two different vectors under conditions sufficient to insert all or a portion of the product of (h) into the vectors.

25. A method according to claim 24, comprising selecting for one of the vectors.
